(19) United States
(12) Patent Application Publication
(10) Pub. No.: US 2003/0165854 A1
(43) Pub. Date: Sep. 4, 2003

(54) Marker genes responding to treatment with toxins

(76) Inventors: Mary Jane Cunningham, The Woodlands, TX (US); Matthew R. Kaser, Castro Valley, CA (US)

Correspondence Address: INCYTE CORPORATION (formerly known as Incyte, Genomics, Inc.), 3160 PORTER DRIVE, PALO ALTO, CA, 94304, US

(21) Appl. No.: 10/006,285

(22) Filed: Dec. 5, 2001

Related U.S. Application Data
(60) Provisional application No. 60/251,986, filed on Dec. 5, 2000.

(51) Int. Cl. C12Q 1/68; C07H 21/04

(52) U.S. Cl. 435/6; 536/23.2

(57) ABSTRACT

The present invention relates to a combination comprising a plurality of cDNAs which are differentially expressed in treated rat liver and in human liver and which may be used in their entirety or in part to diagnose, stage, or treat a liver disorder, to monitor diagnostic and therapeutic applications, to detect metabolic and toxicological responses, and to elucidate drug mechanism of action.

This application is a nonprovisional application which hereby claims the benefit under Title 35, United States Code § 119(e) of U.S. provisional application Serial No. 60/251,986 filed Dec. 5, 2000.

### FIELD OF THE INVENTION

The present invention relates to a combination comprising a plurality of cDNAs which are differentially expressed in liver treated with toxins and which may be used entirely or in part to diagnose, stage, or treat a liver disorder, to monitor diagnostic and therapeutic applications, to detect metabolic and toxicological responses, and to elucidate drug mechanism of action.

### BACKGROUND OF THE INVENTION

Toxicity testing is a mandatory and time-consuming part of drug development programs in the pharmaceutical industry. A more rapid screen to determine the effects upon metabolism and to detect toxicity of lead drug candidates may be the use of gene expression microarrays. For example, microarrays of various kinds may be produced using full length genes or gene fragments. These arrays can then be used to test samples treated with the drug candidates to elucidate the gene expression pattern associated with drug treatment. This gene pattern can be compared with gene expression patterns associated with compounds which produce known metabolic and toxicological responses.

Benzo(a)pyrene is a known rodent and likely human carcinogen and is the prototype of a class of compounds, the polycyclic aromatic hydrocarbons (PAH). It is metabolized by several forms of cytochrome P450 (P450 isozymes) and associated enzymes to form both activated and detoxified metabolites. The ultimate metabolites are the bay-region diol epoxide, benzo(a)pyrene-7,8-diol-9,10-epoxide (BPDE) and the K-region diol epoxide, 9-hydroxy benzo(a)pyrene-4,5-oxide, both of which induce formation of DNA adducts. DNA adducts have been shown to persist in rat liver up to 56 days following treatment with benzo(a)pyrene at a dose of 10 mg/kg body weight three times per week for two weeks (Qu and Stacey (1996) Carcinogenesis 17:53-59).

Acetaminophen is a widely-used analgesic. It is metabolized by specific cytochrome P450 isozymes with the majority of the drug undergoing detoxification by glucuronic acid, sulfate and glutathione conjugation pathways. However, at supratherapeutic doses, acetaminophen is metabolized to an active intermediate, N-acetyl-p-benzoquinone imine (NAPQI) which can cause hepatic and renal failure. NAPQI then binds to sulfhydryl groups of proteins causing their inactivation and leading to subsequent cell death (Kroger et al. (1997) Gen Pharmacol 28:257-263).

Clofibrate is an hypolidemic drug which lowers elevated levels of serum triglycerides. In rodents, chronic treatment produces hepatomegaly and an increase in hepatic peroxisomes (peroxisome proliferation). Peroxisome proliferators (PPs) are a class of drugs which activate the PP-activated receptor in rodent liver, leading to enzyme induction, stimulation of S-phase, and a suppression of apoptosis (Hasmall and Roberts (1999) Pharmacol Ther 82:63-70). PPs include the fibrate class of hypolipidemic drugs, phenobarbitone, thiazolidinediones, certain non-steroidal anti-inflammatory drugs, and naturally-occuring fatty acid-derived molecules (Gelman et al. (1999) Cell Mol Life Sci 55:932-943). Clofibrate has been shown to increase levels of cytochrome P450 4A. It is also involved in transcription of β-oxidation genes as well as induction of PP-activated receptors (Kawashima et al. (1997) Arch Biochem Biophys 347:148-154). Peroxisome proliferation that is induced by both clofibrate and the chemically-related compound fenofibrate is mediated by a common inhibitory effect on mitochondrial membrane depolarization (Zhou and Wallace (1999) Toxicol Sci 48:82-89).

Toxicological effects in the liver are also induced by other compounds. These can include carbon tetrachloride (a necrotic agent), hydrazine (a steatotic agent), α-naphthylisothiocyanate (a cholestatic agent), 4-acetylaminofluorene (a liver mitogen), 3-methylcholanthrene (a potent carcinogen) and their corresponding metabolites which are used in experimental protocols to measure toxicological responses (Waterfield et al. (1993) Arch Toxicol 67:244-254).

The Han/Wistar strain of the Norway rat was found to be particularly resistant to the highly toxic environmental contaminant 2,3,7,8-tetrachlorodibenzo-p-dioxin (TCDD) compared to the Long-Evans strain of rat. Studies of liver-derived serum lipid and carbohydrate parameters and circulating regulatory hormones suggested that liver metabolic processes in response to a toxin are modulated by multifactoral genetic and epigenetic events and were therefore not predictable. For example, cytosolic levels of the aromatic hydrocarbon receptor (AHR), which is believed to mediate the toxic effects of TCCD, was significantly reduced in livers from the Han/Wistar strain. However, most alterations caused by TCDD were similar in both strains. Given that an experimental animal model in which an otherwise lethal response may be studied is available, the Han/Wistar is frequently the strain of choice to better understand the response of the liver to a toxic challenge (Pohjanvirta et al. (1987) Pharmacol Toxicol 60:145-150; ibid (1987) Arch Toxicol Suppl 11:345-347; ibid (1990) Pharmacol Toxicol 66:399-408; ibid (1998) Fundam Appl Toxicol 12:698-712; and ibid (1999) Toxicol Appl Pharmacol 155:82-95).

The potential application of gene expression profiling is particularly relevant to improving diagnosis, prognosis, and treatment of disease. For example, the amount of expression of a large number of cDNAs in tissues from subjects with biliary cirrhosis may be compared with the amount of expression of those cDNAs in normal tissue.

The present invention provides cDNAs and protein molecules which satisfy a need in the art for molecules which can be used to detect, treat and monitor the treatment of liver disorders, to provide measurements of metabolic and toxicological responses following treatment with known and experimental drugs, and in elucidate drug mechanisms of action.

### SUMMARY

The present invention provides a combination comprising a plurality of cDNAs and their complements which are differentially expressed in liver tissues in response to treatment with a toxin and which are selected from SEQ ID NOs: 1-514 as presented in the Sequence Listing. In one embodiment, each cDNA is differentially expressed at least three-fold, SEQ ID NOs: 1-202; in another embodiment, each cDNA is a rat template, SEQ ID NOs: 203-399; in yet another embodiment, each cDNA is a human cDNA homolgous to a rat cDNA, SEQ ID NOs: 400-514. In still yet another embodiment each human cDNA, SEQ ID NOs: 402, 404, 405, 407, 416, 421, 428, 431, 435, 438, 441, 446, 448, 456-458, 475, 489, 501, 502, 512, and 514, is predominantly expressed in liver. In one aspect, the combination is useful to diagnose a liver disorder selected from biliary cirrhosis, X-inked adrenoleukodystrophy, Zellweger syndrome, hepatorenal syndrome, hepatitis, and hepatocarcinoma. In another aspect, the combination is immobilized on a substrate.

The invention also provides a high throughput method to detect differential expression of one or more of the cDNAs of the combination. The method comprises hybridizing the substrate containing the combination with the nucleic acids of a sample, thereby forming one or more hybridization complexes, detecting the hybridization complexes, and comparing the hybridization complexes with those of a standard, wherein differences in the size and signal intensity of each hybridization complex indicates differential expression of nucleic acids in the sample. In one aspect, the sample is from a subject treated with a drug, and differential expression is used to evaluate the response to that treatment.

The invention further provides a high throughput method of screening a library or a plurality of molecules or compounds to identify a ligand. The method comprises combining the substrate containing the combination with the library or plurality of molecules or compounds under conditions to allow and to detect specific binding, thereby identifying a ligand. The library or plurality of molecules or compounds are selected from DNA molecules, RNA molecules, peptide nucleic acid molecules, mimetics, peptides, transcription factors, repressors, and other regulatory proteins. The invention additionally provides a method for purifying a ligand, the method comprising combining a cDNA of the invention with a sample under conditions which allow specific binding, recovering the bound cDNA, and separating the cDNA from the ligand, thereby obtaining purified ligand.

The invention still further provides an isolated cDNA selected from SEQ ID NOs: 411, 432, 441, 450, 457, 465, 474, 477, 499, 501, and 510 as presented in the Sequence Listing. The invention also provides a vector comprising the cDNA, a host cell comprising the vector, and a method for producing a protein comprising culturing the host cell under conditions for the expression of a protein and recovering the protein from the host cell culture.

The present invention provides a purified protein encoded and produced by a cDNA of the invention. The invention also provides a high-throughput method for using a protein to screen a library or a plurality of molecules or compounds to identify a ligand. The method comprises combining the protein or a portion thereof with the library or plurality of molecules or compounds under conditions to allow specific binding and detecting specific binding, thereby identifying a ligand which specifically binds the protein. The library or plurality of molecules or compounds are selected from DNA molecules, RNA molecules, peptide nucleic acid molecules, mimetics, peptides, proteins, agonists, antagonists, antibodies or their fragments, immunoglobulins, inhibitors, drug compounds, and pharmaceutical agents.

The invention further provides a method for using a protein to purify a ligand. The method comprises combining the protein or a portion thereof with a sample under conditions to allow specific binding, recovering the bound protein, and separating the protein from the ligand, thereby obtaining purified ligand. The invention still further provides a pharmaceutical composition comprising the protein. The invention still further provides a method for using the protein to produce an antibody. The method comprises immunizing an animal with the protein or an antigenically-effective epitope under conditions to elicit an antibody response, isolating animal antibodies, and screening the isolated antibodies with the protein to identify an antibody which specifically binds the protein. The invention yet still further provides a method for using the protein to purify antibodies which bind specifically to the protein.

### DESCRIPTION OF THE COMPACT DISC-RECORDABLE (CD-R)

CD-R 1 is labeled: “PA-0039 US, Copy 1,” was created on Dec. 5, 2001 the Sequence Listing formatted in plain ASCII text. The file for the Sequence Listing is entitled pa0039sl.txt, created on Dec. 5, 2001 and is 685 KB in size.

CD-R 2 is an exact copy of CD-R 1. CD-R 2 is labeled: “PA-0039 US, Copy 2,” and was created on Dec. 5, 2001.

The CD-R labeled as: “PA-0039 US, CRF,” contains the Sequence Listing formatted in plain ASCII text. The file for the Sequence Listing is entitled pa0039sl.txt, was created on Dec. 5, 2001 and is 685 KB in size.

The content of the Sequence Listing and each Table named above and as described below, submitted in duplicate on two (2) CD-Rs (labeled “PA-0039 US, Copy 1” and “PA-0039 US, Copy 2”), and the CRF (labeled “PA-0039 US, CRF”) containing the Sequence Listing, are incorporated by reference herein, in their entirety.

### DESCRIPTION OF THE SEQUENCE LISTING AND TABLES

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of cDNAs obtained by sequencing and extension of clone inserts. Each sequence is identified by a sequence identification number (SEQ ID NO) and by the clone or nucleic acid sequence number (Incyte ID) from which it was obtained.

Table 1 lists the abundance of the rat cDNAs of the present invention which were differentially expressed at levels at least three-fold in liver treated with a toxin compared with levels of rat cDNA in untreated liver. Columns 1 and 2 show the SEQ ID NO and Clone ID of the rat cDNA clone, respectively. Column 3 shows the abundance of cDNA clones in rat liver treated with a toxin. Column 4 shows the rat strain name in which expression of the clone is preferentially regulated: Han/Wistar (HW) or Sprague-Dawley (SD). Column 5 shows the gender (F or M) of the rat in which expression of the clone is preferentially regulated. Column 6 shows the treatment whereby expression of the clone is preferentially regulated: acetominophen (APAP), benzo(a)pyrene (BP), clofibrate (CLO), α-naphthylisothiocyanate (ANIT), 4-acetylaminofluorene (4-AAF), hydrazine (Hydra), fenofibrate (Feno), and carbon tetrachloride (CCL4).

Table 2 lists the rat cDNAs (clone), the related rat (Rat Template), and human templates (Human Template) of the present invention. Columns 1 and 2 show the rat clone SEQ ID NO and the related Rat Template SEQ ID NO, respectively. Columns 3 and 4 show the related Human Template Number and Human Template SEQ ID NO, respectively. Columns 5, 6, and 7 show the Genbank ID of the Human Template BLAST Hit, the Hit Description (annotation in Genbank), and the calculated BLAST E-value, respectively.

Table 3 lists the tissue distribution of the human cDNAs. Columns 1 and 2 show the SEQ ID NO and Human Template ID of the human cDNA, respectively. Column 3 shows the tissue distribution of the cDNAs in the LIFESEQ GOLD database (Incyte Genomics, Palo Alto Calif.) as a percentage of all tissues in which cDNAs were found.

### DESCRIPTION OF THE INVENTION

Definitions

“Array” refers to an ordered arrangement of at least two cDNAs, proteins, or antibodies on a substrate. At least one of the cDNAs, proteins, or antibodies represents a control or standard, and the other, a cDNA, protein, or antibody of diagnostic or therapeutic interest. The arrangement of two to about 40,000 cDNAs, proteins, or antibodies on the substrate assures that the size and signal intensity of each labeled complex, formed between each cDNA and at least one nucleic acid, or antibody:protein complex, formed between each antibody and at least one protein to which the antibody specifically binds, is individually distinguishable.

A “combination” refers to at least two and up to 514 cDNAs selected from the group consisting of SEQ ID NOs: 1-514 or their complements as presented in the Sequence Listing.

“cDNA” refers to an isolated polynucleotide, nucleic acid molecule, or any fragment or complement thereof. It may have originated recombinantly or synthetically, may be double-stranded or single-stranded, represents coding and noncoding 3′ or 5′ sequence, and generally lacks introns. It may be combined with carbohydrate, lipids, protein or other materials to perform a particular activity such as diagnosis or form a useful composition for therapy.

The phrase “cDNA encoding a protein” refers to a nucleic acid sequence that closely aligns with sequences which encode conserved regions, motifs or domains that were identified by employing analyses well known in the art. These analyses include BLAST (Basic Local Alignment Search Tool; Altschul (1993) J Mol Evol 36: 290-300; Altschul et al. (1990) J Mol Biol 215:403-410) which provides identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner et al., page 6076, column 2).

The “complement” of a cDNA refers to a nucleic acid molecule which is completely complementary to the cDNA over its full length and which will hybridize to the cDNA or an mRNA under conditions of high stringency.

“Derivative” refers to a cDNA, a protein, or an antibody that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivatization of an antibody involves fragmentation of the antibody and fusion with a peptide or other molecule or agent. Derivative cDNAs, proteins, or antibodies retain the biological activities of the naturally occurring molecule but may confer advantages such as longer lifespan or enhanced activity.

“Differential expression” refers to an increased or up-regulated or a decreased or down-regulated expression as detected by presence, absence or at least two-fold change in the amount or abundance of a transcribed messenger RNA or translated protein in a sample.

“Disorder” refers to a condition, disease or disorder of the liver disorder including, but not limited to, biliary cirrhosis, X-linked adrenoleukodystrophy, Zellweger syndrome, hepatorenal syndrome, hepatitis, and hepatocarcinoma.

“Identity” as applied to nucleic acid or protein sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997) Nucleic Acids Res 25:3389-3402). BLAST2 may be used in a standardized and reproducible way to insert gaps in one of the sequences in order to optimize alignment and to achieve a more meaningful comparison between them. Similarity is an analogous score, but it is calculated with conservative substitutions taken into account; for example, substitution of a valine for a isoleucine or leucine.

“Isolated or purified” refers to a cDNA, protein, or antibody that is removed from its natural environment and that is separated from other components with which it is naturally present.

“Labeling moiety” refers to any reporter molecule, visible or radioactive label, than can be attached to or incorporated into a cDNA, protein or antibody. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Protein” refers to a polypeptide or any portion thereof. A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR).

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a biopsy, a cell; a tissue; a tissue print; a fingerprint, buccal cells, skin, or hair; and the like.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Template” refers to a consensus sequence that was created using the LIFESEQ GOLD database and the assembly algorithm described in U.S. Ser. No. 09/276,534, filed Mar. 25, 1999 and incorporated by reference herein.

A “transcript image” is a profile of gene transcription activity in a particular tissue at a particular time.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

### THE INVENTION

The present invention relates to a combination comprising a plurality of cDNAs, SEQ ID NOs: 1-514 or their complements, which are differentially expressed in liver treated with toxins and which may be used entirely or in part to diagnose, stage, or treat a liver disorder, to monitor diagnostic and therapeutic applications, to detect metabolic and toxicological responses, and to investigate drug mechanism of action. The combination of cDNAs, SEQ ID NOs: 1-202 represent gene transcripts differentially expressed in the cells or tissues of rat liver from subjects treated with a toxin. The combination of cDNAs, SEQ ID NOs: 203-399, are rat templates assembled from cloned transcripts. The combination of cDNAs, SEQ ID NOs: 400-514 are human cDNAs which are homologous to the rat cDNAs and templates. SEQ ID NOs: 402, 404, 405, 407, 416, 421, 428, 431, 435, 438, 441, 446, 448, 456-458, 475, 489, 501, 502, 512, and 514 are predominantly expressed in human liver. SEQ ID NOs: 411, 432, 441, 450, 457, 465, 474, 477, 499, 501, and 510 represent novel human cDNAs. Since the novel human cDNAs were identified solely by the differential expression of their rat orthologs, it is not essential to know a priori the name, structure, or function of the gene or its encoded protein. The usefulness of the novel human cDNAs exists in their immediate value as diagnostics, and for establishing drug efficacy or toxic response.

Table 1 shows cDNAs cloned from expressed transcripts from a rat liver treated with a toxin. The cDNAs from treated libraries have 3-fold or greater (differential) expression compared with expression levels in untreated liver libraries. Toxins include acetominophen (APAP), benzo(a)pyrene (BP), clofibrate (CLO), α-naphthylisothiocyanate (ANIT), 4-acetylaminofluorene (4-AAF), hydrazine (Hydra), fenofibrate (Feno), and carbon tetrachloride (CCL4). Abundance of a clone was determined by counting the number of clones present in a master cluster.

Table 2 lists the rat cDNAs and templates and their related human cDNAs and templates. Where the row is blank, the rat templates had no human ortholog. Human templates which had a Genbank BLASTn E-value between 1 and 1×10⁻⁸ are described as “Incyte Unique”.

Table 3 lists the tissue distribution of the human cDNAs. The category: 1) sense organs, includes fetal cochlea; corneal fibroblasts primary line; corneal stroma primary line; pooled fetal retina; adult retina; and olfactory epithelium; and 2) unclassified/mixed, includes mixed melanocytes, uterus, fetal heart, fetal lung, testis, B-cells, pooled metastatic breast and ovary, pooled metastatic brain and Wilms tumor, pooled mixed sarcoma, myometrium, smooth muscle cells, and pooled high-grade urogenital transitional cell carcinoma. Where tissue distribution specificity was less than 10% percent, the distribution was classified “widely distributed”.

As shown in Table 1, rat cDNAs, SEQ ID NOs: 1-202, were identified as having greater than three-fold increased gene transcript levels in liver cDNA libraries isolated from rats treated with toxins compared with cDNAs from untreated rat liver libraries. These cDNAs were represented by at least three distinct clones corresponding to a single mRNA species.

In particular, specific mRNA species, SEQ ID NOs: 1-61, were upregulated only in cDNA libraries isolated from Han/Wistar rat liver whereas specific mRNA species, SEQ ID NOs: 62-202, were upregulated only in cDNA libraries isolated from Sprague Dawley rat liver. SEQ ID NOs: 1-61 may therefore be involved with molecular pathways which confer particular resistance to toxins in the Han/Wistar rat. SEQ ID NOs: 62-202 may therefore be involved with molecular pathways which confer particular resistance to toxins in the Sprague Dawley rat.

As shown in Table 1, levels of SEQ ID NOs: 1-11 may be preferentially upregulated in response to treatment with APAP, B(a)P, both B(a)P and CLO, and CLO, respectively. Messenger RNA levels of SEQ ID NOs: 12-61 were upregulated by more than two toxins and may therefore be involved with a broader metabolic response to toxic insult in the Han/Wistar rat. In addition, gender specific expression was seen in the mRNA levels of SEQ ID NO: 10 and in SEQ ID NOs: 12-14, which were predominantly upregulated in male and female rats, respectively.

Similarly, and also shown in Table 1, specific mRNA species of SEQ ID NOs: 62-202 were upregulated only in cDNA libraries isolated from Sprague Dawley rat liver treated with toxins. Moreover, the mRNA species were predominantly upregulated only in response to certain toxins: SEQ ID NOs: 62-73 by APAP; SEQ ID NOs: 74-106 by B(a)P; SEQ ID NOs: 107, 108, and 202 by APAP and B(a)P; SEQ ID NOs: 109-112, and 186-188, 190-191, 197, and 201 by B(a)P and CLO; SEQ ID NOs: 113-185, 189, 192, 194, 195, 198, 199 by CLO; and SEQID NOs: 193, 196, and 200 by more than two toxins. SEQ ID NOs: 193, 196, and 200 may be involved with a broader metabolic response to toxic insult in the Sprague Dawley rat liver. In addition gender specific expression was seen in the mRNA levels of SEQ ID NOs: 62-64, 109, 126-162, and 194-200 which were predominantly upregulated in male rats and in the mRNA levels of SEQ ID NOs: 74, 75, 113-125, and 188-193 which were predominantly upregulated in female rats.

The results in Table 1 appear to show a bias towards rat mRNAs responding to treatment with CLO, but this is due to an analytical artifact, since a large proportion of the cDNA libraries included in the analysis were constructed following subtractive hybridization of CLO-treated parent cDNA libraries.

As shown in Table 2, SEQ ID NOs: 400-514 are human homologs of the respective rat sequences, SEQ ID NOs: 176, 8, 7, 181, 32, 72, 109, 1, 188, 190, 14, 29, 27, 117, 6, 12, 196, 42, 189, 37, 100, 200, 78, 119, 23, 13, 41, 68, 5, 82, 175, 80, 39, 56, 137, 193, 16, 131, 90, 201, 17, 38, 87, 202, 177, 59, 158, 111, 47, 173, 11, 128, 79, 19, 48, 134, 43, 165, 192, 4, 15, 99, 84, 36, 163, 35, 33, 55, 143, 92, 168, 166, 126,40, 130, 115, 91, 191, 118, 63, 187, 77, 31, 172, 81, 174, 141, 46, 135, 51, 28, 159, 125, 184, 104, 45, 24, 65, 197, 34, 199, 94, 195, 129, 83, and 22, respectively, identified by BLAST analysis using the LIFESEQ GOLD database (Incyte Genomics). In addition, SEQ ID NOs: 411, 441, 432, 450, 457, 465, 474, 477, 499, 501, and 510 are novel human cDNAs.

As shown in Table 3, SEQ ID NOs: 402-405, 407, 416, 421, 428, 431, 435, 438, 441, 446, 448, 456-458, 475, 489, 501, 502, 512, and 514 are found predominantly in 22 human liver cDNA libraries from a total of 115 tissues (19%). The human liver cDNA libraries include tissue derived from fetal liver, livers with the following diagnosed pathologies: primary biliary cirrhosis, metastatic neuroendocrine carcinoma, hepatoma, metastatic colon adenocarcinoma, adenoma, hepatitis C, and from the human C3A hepatocyte cell line treated with APAP, pentobarbital, 3-methylcholanthrene (MCA), or vehicle. In addition, SEQ ID NOs: 441 and 457 are novel human cDNAs expressed in the C3A cell line treated with 5 mM MCA for 48 hours.

As shown in Table 1, SEQ ID NO: 441 is the human homolog of a rat cDNA which is differentially expressed in the Han/Wistar strain in response to treatment with toxins including carbon tetrachloride, α-naphthylisothiocyanate, and 4-acetylaminofluorene; and SEQ ID NO: 457 is the human homolog of a rat cDNA which is differentially expressed in male Sprague-Dawley rats in response to clofibrate treatment.

The cDNAs of the invention define a differential expression pattern against which to compare the expression pattern of biopsied and/or in vitro treated liver tissues. Experimentally, differential expression of the cDNAs can be evaluated by methods including, but not limited to, differential display by array technologies, clustering, gel electrophoresis and mass spectrophotometric analysis, genome mismatch scanning, representational discriminant analysis, spatial immobilization or transcript imaging. These methods may be used alone or in combination.

The combination may be arranged on a substrate and hybridized with samples from subjects with diagnosed liver disorders to identify those sequences which are differentially expressed in biliary cirrhosis and/ or other liver disorders. This process allows identification of those sequences of highest diagnostic and potential therapeutic value for a particular disorder. In one embodiment, an additional set of cDNAs, such as cDNAs encoding signaling molecules, are arranged on the substrate with the combination. Such combinations may be useful in the elucidation of pathways which are affected in a particular liver disorder or to identify new, coexpressed, candidate, therapeutic molecules.

In another embodiment, the combination can be used for large scale genetic or gene expression analysis of a large number of novel, nucleic acid molecules. These samples are prepared by methods well known in the art and are from mammalian cells or tissues which are in a certain stage of development; have been treated with a known molecule or compound, such as a cytokine, growth factor, a drug, and the like; or have been extracted or biopsied from a mammal with a known or unknown condition, disorder, or disease before or after treatment. The sample nucleic acids are hybridized to the combination for the purpose of defining a novel gene profile that is specifically associated with a particular disorder, developmental stage, or treatment protocol.

cDNAs and Their Uses

cDNAs can be prepared by a variety of synthetic or enzymatic methods well known in the art. cDNAs can be synthesized, in whole or in part, using chemical methods well known in the art (Caruthers et al. (1980) Nucleic Acids Symp Ser (7)215-233). Alternatively, cDNAs can be produced enzymatically or recombinantly, by in vitro or in vivo transcription.

Nucleotide analogs can be incorporated into cDNAs by methods well known in the art. The only requirement is that the incorporated analog must base pair with native purines or pyrimidines. For example, 2, 6-diaminopurine can substitute for adenine and form stronger bonds with thymidine than those between adenine and thymidine. A weaker pair is formed when hypoxanthine is substituted for guanine and base pairs with cytosine. Additionally, cDNAs can include nucleotides that have been derivatized chemically or enzymatically.

cDNAs can be synthesized on a substrate. Synthesis on the surface of a substrate may be accomplished using a chemical coupling procedure and a piezoelectric printing apparatus as described by Baldeschweiler et al. (PCT publication WO95/251116). Alternatively, the cDNAs can be synthesized on a substrate surface using a self-addressable electronic device that controls when reagents are added as described by Heller et al. (U.S. Pat. No. 5,605,662). cDNAs can be synthesized directly on a substrate by sequentially dispensing reagents for their synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

cDNAs can be immobilized on a substrate by covalent means such as by chemical bonding procedures or UV irradiation. In one method, a cDNA is bound to a glass surface which has been modified to contain epoxide or aldehyde groups. In another method, a cDNA is placed on a polylysine coated surface and UV cross-linked to it as described by Shalon et al. (WO95/35505). In yet another method, a cDNA is actively transported from a solution to a given position on a substrate by electrical means (Heller, supra). cDNAs do not have to be directly bound to the substrate, but rather can be bound to the substrate through a linker group. The linker groups are typically about 6 to 50 atoms long to provide exposure of the attached cDNA. Preferred linker groups include ethylene glycol oligomers, diamines, diacids and the like. Reactive groups on the substrate surface react with a terminal group of the linker to bind the linker to the substrate. The other terminus of the linker is then bound to the cDNA. Alternatively, polynucleotides, plasmids or cells can be arranged on a filter. In the latter case, cells are lysed, proteins and cellular components degraded, and the DNA is coupled to the filter by UV cross-linking.

The cDNAs may be used for a variety of purposes. For example, the combination of the invention may be used on an array. The array, in turn, can be used in high-throughput methods for detecting an identical or related polynucleotide in a sample, screening a plurality of molecules or compounds to identify a ligand, diagnosing a liver disorder, or inhibiting or inactivating a therapeutically relevant gene related to the cDNA.

When the cDNAs of the invention are employed on a microarray, the cDNAs are arranged in an ordered fashion so that each cDNA is present at a specified location. Because the cDNAs are at specified locations on the substrate, the hybridization patterns and intensities, which together create a unique expression profile, can be interpreted in terms of expression levels of particular genes and can be correlated with a particular metabolic process, condition, disorder, disease, stage of disease, or treatment. Hybridization

The cDNAs or fragments or complements thereof may be used in various hybridization technologies. The cDNAs may be labeled using a variety of reporter molecules by either PCR, recombinant, or enzymatic techniques. For example, a commercially available vector containing the cDNA is transcribed in the presence of an appropriate polymerase, such as T7 or SP6 polymerase, and at least one labeled nucleotide. Commercial kits are available for labeling and cleanup of such cDNAs. Radioactive (Amersham Pharmacia Biotech (APB), Piscataway N.J.), fluorescent (Operon Technologies, Alameda Calif.), and chemiluminescent labeling (Promega, Madison Wis.) are well known in the art.

A cDNA may represent the complete coding region of an mRNA or be designed or derived from unique regions of the mRNA or genomic molecule, an intron, a 3′ untranslated region, or from a conserved motif. The cDNA is at least 18 contiguous nucleotides in length and is usually single stranded. Such a cDNA may be used under hybridization conditions that allow binding only to an identical sequence, a naturally occurring molecule encoding the same protein, or an allelic variant. Discovery of related human and mammalian sequences may also be accomplished using a pool of degenerate cDNAs and appropriate hybridization conditions. Generally, a cDNA for use in Southern or northern hybridizations may be from about 400 to about 6000 nucleotides long. Such cDNAs have high binding specificity in solution-based or substrate-based hybridizations. An oligonucleotide, a fragment of the cDNA, may be used to detect a polynucleotide in a sample using PCR.

The stringency of hybridization is determined by G+C content of the cDNA, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5× saline sodium citrate (SSC) with 1% sodium dodecyl sulfate (SDS) at 60° C., that permit the formation of a hybridization complex between nucleic acid sequences that contain some mismatches. Subsequent washes are performed with buffers such as 0.2×SSC with 0.1 % SDS at either 45° C. (medium stringency) or 65°-68° C. (high stringency). At high stringency, hybridization complexes will remain stable only where the nucleic acid molecules are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or Triton X-100 (Sigma-Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4.6-4.9).

Dot-blot, slot-blot, low density and high density arrays are prepared and analyzed using methods known in the art. cDNAs from about 18 consecutive nucleotides to about 5000 consecutive nucleotides in length are contemplated by the invention and used in array technologies. The preferred number of cDNAs on an array is at least about 100,000, a more preferred number is at least about 40,000, an even more preferred number is at least about 10,000, and a most preferred number is at least about 600 to about 800. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; WO95/11995; WO95/35505; U.S. Pat. No. 5,605,662; and U.S. Pat. No. 5,958,342.)

Screening and Purification Assays

A cDNA may be used to screen a library or a plurality of molecules or compounds for a ligand which specifically binds the cDNA. Ligands may be DNA molecules, RNA molecules, peptide nucleic acid molecules, peptides, proteins such as transcription factors, promoters, enhancers, repressors, and other proteins that regulate replication, transcription, or translation of a gene in a biological system. The assay involves combining the cDNA or a fragment thereof with the molecules or compounds under conditions that allow specific binding and detecting the bound cDNA to identify at least one ligand that specifically binds the cDNA.

In one embodiment, the cDNA may be incubated with a library of isolated and purified molecules or compounds and binding activity determined by methods such as a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay. Protein binding may be confirmed by raising antibodies against the protein and adding the antibodies to the gel-retardation assay where specific binding will cause a supershift in the assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

The cDNA may be used to purify a ligand from a sample. A method for using a cDNA to purify a ligand would involve combining the cDNA or a fragment thereof with a sample under conditions to allow specific binding, recovering the bound cDNA, and using an appropriate agent to separate the cDNA from the purified ligand.

Protein Production and Uses

The full length cDNAs or fragment thereof may be used to produce purified proteins using recombinant DNA technologies described herein and taught in Ausubel et al. (supra; Units 16.1-16.62). One of the advantages of producing proteins by these procedures is the ability to obtain highly-enriched sources of the proteins thereby simplifying purification procedures.

The proteins may contain amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.), MACVECTOR software (Genetics Computer Group, Madison Wis.) and RasMol software (University of Massachusetts, Amherst Mass.) may be used to help determine which and how many amino acid residues in a particular portion of the protein may be substituted, inserted, or deleted without abolishing biological or immunological activity.

Expression of Encoded Proteins

Expression of a particular cDNA may be accomplished by cloning the cDNA into a vector and transforming this vector into a host cell. The cloning vector used for the construction of cDNA libraries in the LIFESEQ databases may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into competent E. coli cells. Induction of the isolated bacterial strain with isopropylthiogalactoside (IPTG) using standard methods will produce a fusion protein that contains an N terminal methionine, the first seven residues of β-galactosidase, about 15 residues of linker, and the protein encoded by the cDNA.

The cDNA may be shuttled into other vectors known to be useful for expression of protein in specific hosts. Oligonucleotides containing cloning sites and fragments of DNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the protein and the purification domain may also be used to recover the protein.

Suitable host cells may include, but are not limited to, mammalian cells such as Chinese Hamster Ovary (CHO) and human 293 cells, insect cells such as Sf9 cells, plant cells such as Nicotiana tabacum, yeast cells such as Saccharomyces cerevisiae, and bacteria such as E. coli. For each of these cell systems, a useful vector may also include an origin of replication and one or two selectable markers to allow selection in bacteria as well as in a transformed eukaryotic host. Vectors for use in eukaryotic host cells may require the addition of 3′ poly(A) tail if the cDNA lacks poly(A).

Additionally, the vector may contain promoters or enhancers that increase gene expression. Many promoters are known and used in the art. Most promoters are host specific and exemplary promoters includes SV40 promoters for CHO cells; T7 promoters for bacterial hosts; viral promoters and enhancers for plant cells; and PGH promoters for yeast. Adenoviral vectors with the rous sarcoma virus enhancer or retroviral vectors with long terminal repeat promoters may be used to drive protein expression in mammalian cell lines. Once homogeneous cultures of recombinant cells are obtained, large quantities of secreted soluble protein may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, proteins or portions thereof may be produced manually, using solid-phase techniques (Stewart et al. (1969) Solid-Phase Peptide Synthesis, W H Freeman, San Francisco Calif.; Merrifield (1963) J Am Chem Soc 5:2149-2154), or using machines such as the ABI 431A peptide synthesizer (Applied Biosystems (ABI), Foster City Calif.). Proteins produced by any of the above methods may be used as pharmaceutical compositions to treat disorders associated with null or inadequate expression of the genomic sequence.

Production of Antibodies

A protein encoded by a cDNA of the invention may be used to produce specific antibodies. Antibodies may be produced using an oligopeptide or a portion of the protein with inherent immunological activity. Methods for producing antibodies include: 1) injecting an animal, usually goats, rabbits, or mice, with the protein, or an antigenically-effective portion or an oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the proteins of the invention are useful for the diagnosis of prepathologic disorders as well as the diagnosis of chronic or acute diseases characterized by abnormalities in the expression, amount, or distribution of the protein. A variety of protocols for competitive binding or immunoradiometric assays using either polyclonal or monoclonal antibodies specific for proteins are well known in the art. Immunoassays typically involve the formation of complexes between a protein and its specific binding molecule or compound and the measurement of complex formation. Immunoassays may employ a two-site, monoclonal-based assay that utilizes monoclonal antibodies reactive to two noninterfering epitopes on a specific protein or a competitive binding assay (Pound (1998) Immunochemical Protocols, Humana Press, Totowa N.J.).

Immunoassay procedures may be used to quantify expression of the protein in cell cultures, in subjects with a particular disorder or in model animal systems under various conditions. Increased or decreased production of proteins as monitored by immunoassay may contribute to knowledge of the cellular activities associated with developmental pathways, engineered conditions or diseases, or treatment efficacy. The quantity of a given protein in a given tissue may be determined by performing immunoassays on freeze-thawed detergent extracts of biological samples and comparing the slope of the binding curves to binding curves generated by purified protein.

Labeling of Molecules for Assay

A wide variety of reporter molecules and conjugation techniques are known by those skilled in the art and may be used in various cDNA, polynucleotide, protein, peptide or antibody assays. Synthesis of labeled molecules may be achieved using commercial kits for incorporation of a labeled nucleotide such as ³²P-dCTP, Cy3-dCTP or Cy5-dCTP or amino acid such as ³⁵S-methionine. Polynucleotides, cDNAs, proteins, or antibodies may be directly labeled with a reporter molecule by chemical conjugation to amines, thiols and other groups present in the molecules using reagents such as BIODIPY or FITC (Molecular Probes, Eugene Oreg.).

The proteins and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. Nos. 3,817,837; 3,850,752; 3,939,350; 3,996,345; 4,277,437; 4,275,149; and 4,366,241.

Diagnostics

The cDNAs, or fragments thereof, may be used to detect and quantify differential gene expression; absence, presence, or excess expression of mRNAs; or to monitor mRNA levels during therapeutic intervention. Disorders associated with altered expression include biliary cirrhosis, X-linked adrenoleukodystrophy, Zellweger syndrome, hepatorenal syndrome, hepatitis, and hepatocarcinoma. These cDNAs can also be utilized as markers of treatment efficacy against the disorders noted above and other disorders, conditions, and diseases over a period ranging from several days to months. The diagnostic assay may use hybridization or amplification technology to compare gene expression in a biological sample from a patient to standard samples in order to detect altered gene expression. Qualitative or quantitative methods for this comparison are well known in the art.

For example, the cDNA may be labeled by standard methods and added to a biological sample from a patient under conditions for hybridization complex formation. After an incubation period, the sample is washed and the amount of label (or signal) associated with hybridization complexes is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of molecule is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

Gene Expression Profiles A gene expression profile comprises a plurality of cDNAs and a plurality of detectable hybridization complexes, wherein each complex is formed by hybridization of one or more probes to one or more complementary sequences in a sample. The cDNAs of the invention are used as elements on a microarray to analyze gene expression profiles. In one embodiment, the microarray is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the microarray is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease; or treatment of the condition, disorder or disease. Novel treatment regimens may be tested in these animal models using microarrays to establish and then follow expression profiles over time. In addition, microarrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against epitopes on a protein encoded by a cDNA of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the protein and its specific antibody and the measurement of such complexes. These and other assays are described in Pound (supra). The method may employ a two-site, monoclonal-based immunoassay utilizing monoclonal antibodies reactive to two non-interfering epitopes, or a competitive binding assay. (See, e.g., Coligan et al. (1997) Current Protocols in Immunology, Wiley-Interscience, New York N.Y.; Pound, supra.)

Therapeutics

The cDNAs of the invention can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of an endogenous gene, reduction or loss of an endogenous protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

In addition, expression of a particular protein can be regulated through the specific binding of a fragment of a cDNA to a genomic sequence or an mRNA which encodes the protein or directs its transcription or translation. The cDNA can be modified or derivatized to any RNA-like or DNA-like material including peptide nucleic acids, branched nucleic acids, and the like. These molecules can be produced biologically by transforming an appropriate host cell with a vector containing the cDNA of interest.

Molecules which regulate the activity of the cDNA or encoded protein are useful as therapeutics for biliary cirrhosis, X-linked adrenoleukodystrophy, Zellweger syndrome, hepatorenal syndrome, hepatitis, and hepatocarcinoma. Such molecules include agonists which increase the expression or activity of the cDNA or encoded protein, respectively; or antagonists which decrease expression or activity of the cDNA or encoded protein, respectively. In one aspect, an antibody which specifically binds the protein may be used directly as an antagonist or indirectly as a delivery mechanism for bringing a pharmaceutical agent to cells or tissues which express the protein.

Additionally, any of the proteins, or their ligands, or complementary nucleic acid sequences may be administered as pharmaceutical compositions or in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration used by doctors and pharmacists may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

Transgenic Animal Models

Transgenic rodents that overexpress or underexpress a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells such as the mouse 129/SvJ cell line are placed in a blastocyst from the C57BL/6 mouse strain, they resume normal development and contribute to tissues of the live-born animal. ES cells are preferred for use in the creation of experimental knockout and knockin animals. The method for this process is well known in the art and the steps are: the cDNA is introduced into a vector, the vector is transformed into ES cells, transformed cells are identified and microinjected into mouse cell blastocysts, blastocysts are surgically transferred to pseudopregnant dams. The resulting chimeric progeny are genotyped and bred to produce heterozygous or homozygous strains.

Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene.

Knockin Analysis

ES cells can be used to create knockin humanized animals or transgenic animal models of human diseases. With knockin technology, a region of a human gene is injected into animal ES cells, and the human sequence integrates into the animal cell genome. Transgenic progeny or inbred lines are studied and treated with potential pharmaceutical agents to obtain information on the progression and treatment of the analogous human condition.

As described herein, the uses of the cDNAs, provided in the Sequence Listing of this application, and their encoded proteins are exemplary of known techniques and are not intended to reflect any limitation on their use in any technique that would be known to the person of average skill in the art. Furthermore, the cDNAs provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining or assembling full length cDNAs that will be known to those skilled in the art. It is also to be understood that this invention is not limited to the particular methodology, protocols, and reagents described, as these may vary. It is also understood that the terminology used herein is for the purpose of describing particular embodiments only, and is not intended to limit the scope of the present invention which will be limited only by the appended claims. The examples below are provided to illustrate the subject invention and are not included for the purpose of limiting the invention.

### EXAMPLES

I cDNA Library Construction

The RALINOT01 cDNA library was constructed from liver tissue removed from a pool of fifty 10- to 11-week-old SPRAGUE DAWLEY female rats (Pharmacon, Waverly Pa.). The animals were housed in standard laboratory caging and fed PMI-certified Rodent Diet #5002. The animals appeared to be in good health at the time tissue was harvested. The animals were anesthetized by CO₂ inhalation, and then cardiocentesis was performed.

Frozen tissue was homogenized and lysed in TRIZOL reagent (1 g tissue/10 ml TRIZOL; Invitrogen) using a POLYTRON homogenizer (PT-3000; Brinkmann Instruments, Westbury N.Y.). After a brief incubation on ice, chloroform (1:5 v/v) was mixed with the reagent, and then centrifuged at 1,000 rpm. The upper aqueous layer was removed to a fresh tube, and the RNA precipitated with isopropanol, resuspended in DEPC-treated water, and treated with DNAse I for 25 min at 37 C. The RNA was re-extracted once with phenol-chloroform, pH 4.7, and precipitated using 0.3 M sodium acetate and 2.5 volumes ethanol. The mRNA was then isolated using an OLIGOTEX kit (Qiagen, Chatsworth Calif.) and used to construct the cDNA library.

The mRNA was handled according to the recommended protocols in the SUPERSCRIPT plasmid system ( Invitrogen). The cDNAs were fractionated on a SEPHAROSE CL-4B column (APB), and those cDNAs exceeding 400 bp were ligated into the pINCY1 plasmid vector (Incyte Genomics). The plasmid pINCY1 was subsequently transformed into DH5α or DH10B competent cells (Invitrogen).

The RAKINOT01 library was constructed using mRNA isolated from kidney tissue removed from a pool of fifty, 7- to 8-week-old male Sprague-Dawley rats, as described above.

The RAKINOT02 library was constructed using mRNA isolated from kidney tissue removed from a pool of fifty, 10- to 11-week-old female Sprague-Dawley rats, as described above.

II cDNA Library Normalization

The RALINOT01 cDNA library was normalized in a single round according to the procedure of Soares et al. (1994, Proc Natl Acad Sci 91:9228-9232) with the following modifications. The primer to template ratio in the primer extension reaction was increased from 2:1 to 10:1. The concentration of each dNTP in the reaction was adjusted to 150 μM to allow for generation of longer (400-1000 nucleotide (nt)) primer extension products and reannealing hybridization was extended from thirteen to forty eight hours. The single stranded DNA circles of the normalized library were purified by hydroxyapatite chromatography, converted to partially double-stranded by random priming, and electroporated into DH10B competent bacteria (Invitrogen).

The Soares normalization procedure is designed to reduce the initial variation in individual cDNA frequencies and to achieve abundances within one order of magnitude while maintaining the overall sequence complexity of the library. In the normalization process, the prevalence of high-abundance cDNA clones decreases significantly, clones with mid-level abundance are relatively unaffected, and clones for rare transcripts are increased in abundance. In the modified Soares normalization procedure, significantly longer hybridization times are used to increase gene discovery rates by biasing the normalized libraries toward low-abundance cDNAs that are well represented in a standard transcript image.

The RALINON03, RALINON04, and RALINON07 normalized rat liver cDNA libraries were constructed with 2.0×10⁶, 4.6×10⁵, and 2.0×10⁶ independent clones from the RALINOT01 cDNA library, respectively.

III cDNA Library Prehybridization

The RALINOH01 cDNA library was constructed with clones from the RALINOT01 cDNA library. After preparation of the RALINOT01 cDNA library, 9,984 clones were spotted onto a nylon filter, lysed, and the plasmid DNA was bound to the filter. The filter was incubated with pre-warmed hybridization buffer and then hybridized at 42 C. for 14-16 hours in 0.75 M NaCl, 0.1 M Na₂HPO₄/NaH₂PO₄, 0.15 M tris-HCl (pH 7.5), 5× Denhardt's Solution, 2% SDS, 100 μg/ml sheared salmon sperm DNA, 50% formamide, and [³²P]-labeled oligonucleotide molecules made from reverse transcribed rat liver mRNA from an untreated animal. The filter was rinsed with 2×SSC at ambient temperature for 5 minutes followed by washing for 30 minutes at 68 C. with pre-warmed washing solution (2×SSC, 1% SDS). The wash was repeated with fresh washing solution for an additional 30 minutes at 68 C. Filters were then washed twice with pre-warmed washing solution (0.6×SSC, 1% SDS) for 30 minutes at 68 C. Some 4,224 clones had very low hybridization signals and about 20% of the clones had no signals and these two groups were isolated and sequenced.

IV Isolation and Sequencing of cDNA Clones

DNA was isolated using the following protocol. Single bacterial colonies were transferred into individual wells of 384-well plates (Genetix Ltd, Christchurch, United Kingdom) using sterile toothpicks.

The wells contained 1 ml of sterile TERRIFIC BROTH (BD Biosciences, San Jose Calif.) with 25 mg/l carbenicillin and 0.4% glycerol (v/v). The plates were covered and placed in an incubator (Thermodyne, Newtown Square Pa.) at 37 C. for 8-10 hours. Plasmid DNA was released from the cells and amplified using direct link PCR (Rao, V. B. (1994) Anal. Biochem. 216:1-14) as follows. The direct link PCR solution included 30 ml of NUCLEIX PLUS PCR nucleotide mix (APB) and 300 μl of Taq DNA polymerase (APB). Five microlitres of the PCR solution were added to each of the 384 wells using the MICROLAB 2200 system (Hamilton, Reno Nev.); plates were centrifuged at 1000 rpm for 20 seconds and refrigerated until use. A 384 pin tool (V&P Scientific, San Diego Calif.) was used to transfer bacterial cells from the incubation plate into the plate containing the PCR solution where 0.1% Tween 20 caused the cells to undergo lysis and release the plasmid DNA. After lysis, the plates were centrifuged up to 500 rpm, covered with a cycle sealer, and cycled using a 384-well DNA ENGINE thermal cycler (M J Research, Watertown Mass.) using the program dPCR30 with the following parameters: Step 1) 95 C., 1 minute; Step 2) 94 C., 30 seconds; Step 3) 55 C., 30 seconds; Step 4) 72 C., 2 minutes; Step 5) steps 2, 3, and 4 repeated 29 times; Step 6) 72 C., 10 minutes; and Step 7) storage at 4 C.

The concentration of DNA in each well was determined by dispensing 100 μl PICO GREEN quantitation reagent (0.25% reagent in 1×TE, v/v; Molecular Probes) and 0.5 μl of undiluted PCR product into each well of an opaque fluorimeter plate (Coming Costar, Acton Mass.) and allowing the DNA to bind to the quantitation reagent. The plate was scanned in a Fluoroscan II (Labsystemns Oy, Helsinki, Finland) to measure the fluorescence of the sample and to quantitate the concentration of DNA. Typical concentrations of each DNA sample were in the range of 100 to 500 ng/ml.

The cDNAs were prepared for sequencing using either a HYDRA microdispenser (Robbins Scientific, Sunnyvale Calif.) or MICROLAB 2200 system (Hamilton) in combination with the DNA ENGINE thermal cyclers (M J Research). The cDNAs were sequenced using the method of Sanger and Coulson (J Mol Biol (1975) 94:441-448) and the ABI PRISM 377 sequencing systems (ABI). Most of the isolates were sequenced according to standard ABI protocols and kits using solution volumes at 0.25×-1.0× concentrations. In the alternative, cDNAs were sequenced using APB solutions and dyes.

V Rat Liver and Kidney Gene Selection

As a first step, originator molecules from high throughput sequencing experiments were derived from clone inserts from RALINOT01, RAKINOT01, RAKINOT02, RALINOH01, RALINON03, RALINON04 and RALINON07. cDNA library clones were obtained. There were 18,140 rat liver molecules and 5,779 rat kidney molecules.

Additionally, 1,500 rat molecules derived from clone inserts of any of 113 rat cDNA libraries were selected based on their homology to genes coding for polypeptides implicated in toxicological responses including peroxisome-associated genes, lysosome-associated genes, apoptosis-associated genes, cytochrome P450 genes, detoxification genes such as sulfotransferases, glutathione S-transferases, and cysteine proteases, and the like. Then, all the remaining molecules derived from all of the rat cDNA library clones were clustered based on the originator molecules described above. The clustering process involved identifying overlapping molecules that have a match quality indicated by a product score of 50 using BLAST. A total of 6581 master clusters were identified.

After clustering, a consensus sequence was produced using PHRAP (Phil Green, University of Washington). The assembled sequences were annotated using BLASTn and GenBank and FASTX and GenPept. About two thirds of the assembled sequences acquired annotation. For nucleic acid sequence analysis, BLASTN 1.4.9MP-WashU was used with default parameters; ctxfactor-2.00; E=10; MatID, 0; Matrix BLOSUM62, +5,−4. For amino acid sequence analysis, NCBI-BLASTX 2.0.4 was used with default parameters; matrix, BLOSUM62; gap penalties, existence 11, extension 1; frameshift window, decay constant 50, 0.1.

VI Treated Rat Liver Library Preparation

Male SPRAGUE DAWLEY rats (6-8 wk old) were dosed intraperitoneally with one of the following: clofibrate (CLO; Acros, Geel, Belgium) at 250 mg/kg body weight (bw); acetaminophen (APAP; Acros) at 1000 mg/kg bw; benzo(a)pyrene (B(a)P; Acros) at 10 mg/kg bw; or dimethylsulfoxide vehicle (DMSO; Acros) at less than 2 ml/kg bw. Animals were monitored daily for physical condition and body weight. Three animals per group were sacrificed approximately 12 hours, 24 hours, 3 days (d), 7d, 14d, and 28d following the single dose. Prior to sacrifice, a blood sample from each animal was drawn and assayed for serum alanine transferase (ALT) and serum aspartate aminotransferase (AST) levels using a diagnostic kit (Sigma-Aldrich). Observed gross pathology and liver weights were recorded at time of necropsy. Liver, kidney, brain, spleen and pancreas from each rat were harvested, flash frozen in liquid nitrogen, and stored at −80 C.

In the alternative, male Han/Wistar rats (8-9 wk old) were dosed by oral gavage with one of the following: fenofibrate (FEN; Sigma-Aldrich) at 250 mg/kg bw; carbon tetrachloride (CCL₄; Sigma-Aldrich) at 3160 mg/kg bw, hydrazine (HYDR; Sigma-Aldrich) at 120 mg/kg bw; α-naphthylisothiocyanate (ANIT; Sigma-Aldrich) at 200 mg/kg bw; 4-acetylaminofluorene (4-AFF; Lancaster Synthesis, Morecambe, Lancashire, UK) at 1000 mg/kg bw; corn oil vehicle or sterile water vehicle at 10 ml/kg bw. The animals were checked twice daily for clinical signs of distress. Blood was collected six days prior to the dose and at sacrifice. Three animals per group were sacrificed approximately six hours and twenty four hours following the single dose. The animals were euthanized by exsanguination under isoflurane anaesthesia. Observed gross pathology and liver weights were recorded at time of necropsy. Livers from each rat were harvested, dissected into approximate 100 mg pieces, flash frozen in liquid nitrogen, and stored at −70 C.

cDNA liver libraries were prepared and sequenced as described above and compared using transcript imaging (TI; Incyte Genomics). Master clusters which contained at least three clones were selected if those clones were present in a treated rat liver cDNA library, no more than one clone was present in an untreated rat liver cDNA library, and the master cluster was not annotated. A master cluster containing at least three clones was given the designation “Abundance=3” and was considered to have been upregulated at least 3-fold.

VII Results

The expression patterns of eight cytochrome P450 isozyme gene transcripts known to be induced in a toxicological response were monitored during a 28 day time course. Expression levels of gene transcripts from liver cDNA libraries from treated rats were compared with those from untreated rats. The results using clofibrate, acetaminophen, and benzo(a)pyrene are shown below in Tables 4, 5, and 6, respectively. Each of the known gene transcripts was upregulated or downregulated greater than 2-fold at least once during the time course as compared with untreated liver as control.

TABLE 4
Gene expression (fold change) of known
genes in clofibrate-treated rat liver

| Gene | 12 hours | 24 hours | 3 days | 7 days | 28 days |
|---|---|---|---|---|---|
| P450 LA-omega | 14.8 | 26.6 | 1.1 | 0.5 | 0.47 |
| 4A3 | | | | | |
| P450 4A | 7.0 | 16.6 | 1.4 | 0.5 | 1.3 |
| P450 3A2 | 0.14 | 1.2 | 0.63 | 0.50 | 0.45 |

TABLE 5
Gene expression (fold change) of known
genes in acetaminophen-treated rat liver

| Gene | 12 hours | 24 hours | 3 days | 7 days | 14 days | 28 days |
|---|---|---|---|---|---|---|
| P450 4A | 1.0 | 4.5 | 2.1 | 2.0 | 4.4 | 4.8 |
| P450f 2C7 | 0.21 | 0.43 | 0.47 | 0.5 | 1.2 | 1.3 |
| P450 | 0.31 | 0.20 | 2.0 | 1.1 | 1.4 | 0.42 |
| 14DM | | | | | | |

TABLE 6
Gene expression (fold change) of known
genes in benzo(a)pyrene-treated rat liver

| | 12 | 24 | 3 | 7 | 14 | 28 |
|---|---|---|---|---|---|---|
| Gene | hours | hours | days | days | days | days |
| P450 LA-omega 4A3 | 1.2 | 2.3 | 2.4 | 1.4 | 6.8 | 1.2 |
| P450 MCA-inducible 1A2 | 7.3 | 9.2 | 5.7 | 2.5 | 2.5 | 0.5 |

Novel cDNAs that are upregulated at least 3-fold at least once during the time course were identified. These cDNAs are SEQ ID NOs: 1-202 provided in the Sequence Listing. These cDNAs can be used to diagnose, stage, or treat a liver disorder, to monitor diagnostic and therapeutic applications, to detect metabolic and toxicological responses, and to elucidate drug mechanism of action.

Table 1 shows the cDNAs that were upregulated at least 3-fold at least once during the time course of treatment with at least one of the following: BP; APAP; CLO; Feno; CCl4; Hydra; ANIT; 4-AFF. Abundance of a particular cDNA in the library profile was determined by summing the total number of copies of the clones present in the master cluster in that library.

VIII Identification and Analyses of Homologous Molecule in other Organisms

The rat cDNAs (SEQ ID NOs: 1-202) identified in the ZOOSEQ v.1.4 (October 1999) database were used to identify related cDNAs and templates in the ZOOSEQ 5.0 and LIFESEQ GOLD databases (Incyte Genomics) which are induced and/or differentially expressed during toxicological response. Table 2 shows the rat and human cDNAs and templates identified using BLAST (SEQ ID NOs: 1-514). Table 3 shows the percentage tissue distribution of the human cDNAs.

IX Construction of Human cDNA Libraries

RNA was purchased from Clontech Laboratories (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Invitrogen). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNAse. For most libraries, poly(A) RNA was isolated using oligo d(T)-coupled paramagnetic particles (Promega), OLIGOTEX latex particles (Qiagen, Valencia Calif.), or an OLIGOTEX mRNA purification kit (Qiagen). Alternatively, poly(A) RNA was isolated directly from tissue lysates using other kits, including the POLY(A)PURE mRNA purification kit (Ambion, Austin Tex.).

In some cases, Stratagene (La Jolla Calif.) was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen) using the recommended procedures or similar methods known in the art. (See Ausubel, supra, Units 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (APB) or preparative agarose gel electrophoresis. cDNAs were ligated into compatible restriction enzyme sites of the polylinker of the pBLUESCRIPT phagemid (Stratagene), pSPORT1 plasmid (Invitrogen), or pINCY plasmid (Incyte Genomics). Recombinant plasmids were transformed into XL1-BLUE, XL1-BLUEMRF, or SOLR competent E. coli cells (Stratagene) or DH5α, DH10B, or ELECTROMAX DH10B competent E. coli cells (Invitrogen).

In some cases, libraries were superinfected with a 5-fold excess of the helper phage, M13K07, according to the method of Vieira et al. (1987, Methods Enzymol 153:3-11) and normalized or subtracted using a methodology adapted from Soares (supra), Swaroop et al. (1991, Nucl Acids Res 19:1954), and Bonaldo et al. (1996, Genome Res 6:791-806). The modified Soares normalization procedure was utilized to reduce the repetitive cloning of highly expressed high abundance cDNAs while maintaining the overall complexity of the library. Modification included significantly longer hybridization times which allowed for increased gene discovery rates by biasing the normalized libraries toward those infrequently expressed low-abundance cDNAs which are poorly represented in a standard transcript image.

X Isolation and Sequencing of cDNA Clones

Plasmids were recovered from host cells by in vivo excision using the UNIZAP vector system (Stratagene) or by cell lysis. Plasmids were purified using one of the following: the Magic or WIZARD MINIPREPS DNA purification system (Promega); the AGTC MINIPREP purification kit (Edge BioSystems, Gaithersburg Md.); the QIAWELL 8, QIAWELL 8 Plus, or QIAWELL 8 Ultra plasmid purification systems, or the REAL PREP 96 plasmid purification kit (Qiagen). Following precipitation, plasmids were resuspended in 0.1 ml of distilled water and stored, with or without lyophilization, at 4 C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes) and a FLUOROSKAN II fluorescence scanner (Labsystems Oy, Helsinki, Finland).

cDNA sequencing reactions were processed using standard methods or high-throughput instrumentation such as the ABI CATALYST 800 thermal cycler (ABI) or the DNA ENGINE thermal cycler (M J Research, Watertown Mass.) in conjunction with the HYDRA microdispenser (Robbins Scientific, Sunnyvale Calif.) or the MICROLAB 2200 system (Hamilton). cDNA sequencing reactions were prepared using reagents provided by APB or supplied in ABI sequencing kits such as the ABI PRISM BIGDYE cycle sequencing kit (ABI). Electrophoretic separation of cDNA sequencing reactions and detection of labeled cDNAs were carried out using the MEGABACE 1000 DNA sequencing system (APB); the ABI PRISM 373 or 377 sequencing systems (ABI) in conjunction with standard ABI protocols and base calling software; or other sequence analysis systems known in the art. Reading frames within the cDNAs were identified using standard methods (reviewed in Ausubel, supra, Unit 7.7).

XI Extension of cDNAs

Nucleic acid sequences were extended using the cDNAs and oligonucleotide primers. One primer was synthesized to initiate 5′ extension of the known fragment, and the other, to initiate 3′ extension of the known fragment. The initial primers were designed using OLIGO primer analysis software (Molecular Biology Insights, Cascade Colo.), or another appropriate program, to be about 22 to 30 nucleotides in length, to have a GC content of about 50% or more, and to anneal to the cDNA at temperatures of about 68 C. to about 72 C. Any stretch of nucleotides which would result in hairpin structures and primer-primer dimerizations was avoided.

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (M J Research). The reaction mix contained DNA template, 200 nmol of each primer, reaction buffer containing Mg²⁺, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B (Incyte Genomics): Step 1: 94 C., 3 min; Step 2: 94 C., 15 sec; Step 3: 60 C., 1 min; Step 4: 68 C., 2 min; Step 5: Steps 2, 3, and 4 repeated 20 times; Step 6: 68 C., 5 min; Step 7: storage at 4 C. In the alternative, the parameters for primer pair T7 and SK+ (Stratagene) were as follows: Step 1: 94 C., 3 min; Step 2: 94 C., 15 sec; Step 3: 57 C., 1 min; Step 4: 68 C., 2 min; Step 5: Steps 2, 3, and 4 repeated 20 times; Step 6: 68 C., 5 min; Step 7: storage at 4 C.

The concentration of DNA in each well was determined using 100 μl PICOGREEN reagent as described above (Molecular Probes). A 5 μl to 10 μl aliquot of the reaction mixture was analyzed by electrophoresis on a 1% agarose mini-gel to determine which reactions were successful in extending the sequence.

The extended nucleic acids were desalted and concentrated, transferred to 384-well plates, digested with CviJI cholera virus endonuclease (Molecular Biology Research, Madison Wis.), and sonicated or sheared prior to religation into pUC18 vector (APB). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC18 vector (APB), treated with Pfu DNA polymerase (Stratagene) to fill-in restriction site overhangs, and transformed into competent E. coli cells. Transformed cells were selected on antibiotic-containing media, and individual colonies were picked and cultured overnight at 37 C. in 384-well plates in LB/2x carbenicillin liquid media.

The cells were lysed, and DNA was amplified by PCR using Taq DNA polymerase (APB) and Pfu DNA polymerase (Stratagene) with the following parameters: Step 1: 94 C., 3 min; Step 2: 94 C., 15 sec; Step 3: 60 C., 1 min; Step 4: 72 C., 2 min; Step 5: steps 2, 3, and 4 repeated 29 times; Step 6: 72 C., 5 min; Step 7: storage at 4 C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the ABI PRISM BIGDYE terminator cycle sequencing kit (ABI).

XII Assembly and Analysis of cDNAs

Component nucleotide sequences from chromatograms were subjected to PHRED analysis (Phil Green, University of Washington, Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs. Sequences were screened using the BLOCK 2 program (Incyte Genomics), a motif analysis program based on sequence information contained in the SWISS-PROT and PROSITE databases (Bairoch et al. (1997) Nucleic Acids Res 25:217-221; Attwood et al. (1997) J Chem Inf Comput Sci 37:417-424).

Processed sequences were subjected to assembly procedures in which the sequences were assigned to bins, one sequence per bin. Sequences in each bin were assembled to produce consensus sequences, nucleic acid sequences. Subsequent new sequences were added to existing bins using BLAST (Altschul (supra); Altschul et al. (supra); Karlin et al. (1988) Proc Natl Acad Sci 85:841-845), BLASTn (vers.1.4, WashU), and CROSSMATCH software (Phil Green, supra). Candidate pairs were identified as all BLAST hits having a quality score greater than or equal to 150. Alignments of at least 82% local identity were accepted into the bin. The component sequences from each bin were assembled using PHRAP (Phil Green, supra). Bins with several overlapping component sequences were assembled using DEEP PHRAP (Phil Green, supra).

Bins were compared against each other, and those having local similarity of at least 82% were combined and reassembled. Reassembled bins having nucleic acid sequences of insufficient overlap (less than 95% local identity) were re-split. Assembled nucleic acid sequences were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to produce the nucleic acid sequences found in the LIFESEQ GOLD database (Incyte Genomics).

The assembled nucleic acid sequences were annotated using the following procedure. Nucleic acid sequences were analyzed using BLASTn (vers. 2.0, NCBI) versus GBpri (GenBank vers. 116). “Hits” were defined as an exact match having from 95% local identity over 200 base pairs through 100% local identity over 100 base pairs, or a homolog match having an E-value equal to or greater than 1×10⁻⁸. (The “E-value” quantifies the statistical probability that a match between two sequences occurred by chance). The hits were subjected to frameshift FASTx versus GENPEPT (GenBank version 109). In this analysis, a homolog match was defined as having an E-value of 1×10⁻⁸. The assembly method used above was described in U.S. Ser. No. 09/276,534, filed Mar. 25, 1999, and the LIFESEQ GOLD user manual (Incyte Genomics).

Following assembly, nucleic acid sequences were subjected to motif, BLAST, Hidden Markov Model (HMM; Pearson and Lipman (1988) Proc Natl Acad Sci 85:2444-2448; Smith and Waterman (1981) J Mol Biol 147:195-197), and functional analyses, and categorized in protein hierarchies using methods described in U.S. Ser. No. 08/812,290, filed Mar. 6, 1997; U.S. Ser. No. 08/947,845, filed Oct. 9, 1997; U.S. Pat. No. 5,953,727; and U.S. Ser. No. 09/034,807, filed Mar. 4, 1998. Nucleic acid sequences may be further queried against public databases such as the GenBank rodent, mammalian, vertebrate, eukaryote, prokaryote, and human EST databases.

XIII Selection of cDNAs, Microarray Preparation and Use

Incyte clones represent all or a portion of nucleic acid sequences derived from the LIFESEQ GOLD assembled human sequence database (Incyte Genomics). In cases where more than one clone is available for a particular nucleic acid sequence, the 5′-most clone is used on the microarray. The GENEALBUM GEM series 1-6 microarrays (Incyte Genomics) contain 52,616 array elements which represent 17,472 annotated clusters and 35,144 unannotated clusters. The HUAN GENOME GEM series 1-3 microarrays (Incyte Genomics) contain 28,626 array elements which represent 10,068 annotated clusters and 18,558 unannotated clusters. For the UNIGEM series microarrays (Incyte Genomics), Incyte clones are mapped to non-redundant Unigene clusters (Unigene database (build 46), NCBI; Shuler (1997) J Mol Med 75:694-698), and the 5′ clone with the strongest BLAST alignment (at least 90% identity and 100 bp overlap) is chosen, verified, and used in the construction of the microarray. The UNIGEM V microarray (Incyte Genomics) contains 7075 array elements which represent 4610 annotated genes and 2,184 unannotated clusters.

To construct microarrays, cDNAs are amplified from transformed bacterial cells using primers complementary to vector sequences flanking the cDNA insert. Thirty cycles of PCR increase the initial quantity of cDNAs from 1-2 ng to a final quantity greater than 5 μg. Amplified cDNAs are then purified using SEPHACRYL-400 columns (APB). Purified cDNAs are immobilized on polymer-coated glass slides. Glass microscope slides (Corning, Corning N.Y.) are cleaned by ultrasound in 0.1% SDS and acetone, with extensive distilled water washes between and after treatments. Glass slides are etched in 4% hydrofluoric acid (VWR Scientific Products, West Chester Pa.), washed thoroughly in distilled water, and coated with 0.05% aminopropyl silane (Sigma Aldrich) in 95% ethanol. Coated slides are cured in a 110 C. oven. cDNAs are applied to the coated glass substrate using a procedure described in U.S. Pat. No. 5,807,522. One microliter of the cDNA at an average concentration of 100 ng/ul is loaded into the open capillary printing element by a high-speed robotic apparatus which then deposits about 5 nl of cDNA per slide.

Microarrays are UV-crosslinked using a STRATALINKER UV-crosslinker (Stratagene), and then washed at room temperature once in 0.2% SDS and three times in distilled water. Non-specific binding sites are blocked by incubation of microarrays in 0.2% casein in phosphate buffered saline (Tropix, Bedford Mass.) for 30 minutes at 60 C. followed by washes in 0.2% SDS and distilled water as before.

XIV Preparation of Samples

Treated tissues or cells are harvested and lysed in 1 ml of TRIZOL reagent (5×10⁶ cells/ml; (Invitrogen). The lysates are vortexed thoroughly and incubated at room temperature for 2-3 minutes and extracted with 0.5 ml chloroform. The extract is mixed, incubated at room temperature for 5 minutes, and centrifuged at 15,000 rpm for 15 minutes at 4° C. The aqueous layer is collected and an equal volume of isopropanol was added. Samples are mixed, incubated at room temperature for 10 minutes, and centrifuged at 15,000 rpm for 20 minutes at 4° C. The supernatant is removed and the RNA pellet is washed with 1 ml of 70% ethanol, centrifuged at 15,000 rpm at 4° C., and resuspended in RNase-free water. The concentration of the RNA is determined by measuring the optical density at 260 nm.

Poly(A) RNA is prepared using an OLIGOTEX mRNA kit (Qiagen) with the following modifications: OLIGOTEX beads are washed in tubes instead of on spin columns, resuspended in elution buffer, and then loaded onto spin columns to recover mRNA. To obtain maximum yield, the mRNA is eluted twice.

Each poly(A) RNA sample is reverse transcribed using MMLV reverse-transcriptase, 0.05 pg/μl oligo-d(T) primer (21 mer), 1× first strand buffer, 0.03 units/ul RNase inhibitor, 500 uM dATP, 500 uM dGTP, 500 uM dTTP, 40 uM dCTP, and 40 uM either dCTP-Cy3 or dCTP-Cy5 (APB). The reverse transcription reaction is performed in a 25 ml volume containing 200 ng poly(A) RNA using the GEMBRIGHT kit (Incyte Genomics). Specific control poly(A) RNAs (YCFR06, YCFR45, YCFR67, YCFR85, YCFR43, YCFR22, YCFR23, YCFR25, YCFR44, YCFR26) are synthesized by in vitro transcription from non-coding yeast genomic DNA (W. Lei, unpublished). As quantitative controls, control mRNAs (YCFR06, YCFR45, YCFR67, and YCFR85) at 0.002ng, 0.02ng, 0.2 ng, and 2ng are diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) are diluted into reverse transcription reaction at ratios of 1:3, 3:1, 1:10, 10:1, 1:25, 25:1 (w/w) to sample mRNA. Reactions are incubated at 37 C. for 2 hr, treated with 2.5 ml of 0.5M sodium hydroxide, and incubated for 20 minutes at 85 C. to the stop the reaction and degrade the RNA.

cDNAs are purified using two successive CHROMA SPIN 30 gel filtration spin columns (Clontech). Cy3- and Cy5-labeled reaction samples are combined as described below and ethanol precipitated using 1 ml of glycogen (1 mg/ml), 60 ml sodium acetate, and 300 ml of 100% ethanol. The cDNAs are then dried to completion using a SpeedVAC system (Savant Instruments, Holbrook N.Y.) and resuspended in 14 μl 5× SSC/0.2% SDS.

XV Hybridization and Detection

Hybridization reactions contain 9 μl of sample mixture containing 0.2 μg each of Cy3 and Cy5 labeled cDNA synthesis products in 5× SSC, 0.2% SDS hybridization buffer. The mixture is heated to 65 C. for 5 minutes and is aliquoted onto the microarray surface and covered with an 1.8 cm² coverslip. The microarrays are transferred to a waterproof chamber having a cavity just slightly larger than a microscope slide. The chamber is kept at 100% humidity internally by the addition of 140 μl of 5× SSC in a corner of the chamber. The chamber containing the microarrays is incubated for about 6.5 hours at 60 C. The microarrays are washed for 10 min at 45 C. in low stringency wash buffer (1× SSC, 0.1% SDS), three times for 10 minutes each at 45 C. in high stringency wash buffer (0.1× SSC), and dried.

Reporter-labeled hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the microarray using a 20× microscope objective (Nikon, Melville N.Y.). The slide containing the microarray is placed on a computer-controlled X-Y stage on the microscope and raster-scanned past the objective. The 1.8 cm×1.8 cm microarray used in the present example is scanned with a resolution of 20 micrometers.

In two separate scans, the mixed gas multiline laser excites the two fluorophores sequentially. Emitted light was split, based on wavelength, into two photomultiplier tube detectors (PMT R1477; Hamamatsu Photonics Systems, Bridgewater N.J.) corresponding to the two fluorophores. Appropriate filters positioned between the microarray and the photomultiplier tubes are used to filter the signals. The emission maxima of the fluorophores used are 565 nm for Cy3 and 650 nm for Cy5. Each microarray is typically scanned twice, one scan per fluorophore using the appropriate filters at the laser source, although the apparatus is capable of recording the spectra from both fluorophores simultaneously.

The sensitivity of the scans is calibrated using the signal intensity generated by a cDNA control species. Samples of the calibrating cDNA are separately labeled with the two fluorophores and identical amounts of each were added to the hybridization mixture. A specific location on the microarray contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood, Mass.) installed in an IBM-compatible PC computer. The digitized data are displayed as an image where the signal intensity is mapped using a linear 20-color transformation to a pseudocolor scale ranging from blue (low signal) to red (high signal). The data are also analyzed quantitatively. Where two different fluorophores are ed and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Significance is defined as signal to background ratio exceeding 2× and area hybridization exceeding 40%.

XVI Data Analysis and Results

Array elements that exhibit at least 3-fold change in expression at one or more time points, a signal intensity over 250 units, a signal-to-background ratio of at least 2.5, and an element spot size of at least 40% are identified as differentially expressed using the GEMTOOLS program (Incyte Genomics). Differential expression values are converted to log base 2 scale. Two-thirds of the cDNAs that were differentially expressed have been annotated using BLAST analysis and GenBank.

XVII Other Hybridization Technologies and Analyses

Other hybridization technologies utilize a variety of substrates. Arranging cDNAs on polymer coated slides was described in Example V; sample cDNA preparation, hybridization, and analysis using polymer coated slides is described in Examples VI and VII, respectively.

The cDNAs are applied to a membrane by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C. for 16 hr. The membrane is removed from the agar and consecutively placed colony side up in 10% SDS, denaturing solution (1.5 M NaCl, 0.5 M NaOH ), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above.

Hybridization probes derived from cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C. for five min and briefly centrifuging. The denatured cDNA is then added to a REDIPRIME tube (APB), gently mixed until blue color is evenly distributed, and briefly centrifuged. Five microliters of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-50 microcolumn (APB). The purified probe is heated to 100 C. for five min and then snap cooled for two min on ice.

Membranes are pre-hybridized in hybridization solution containing 1% Sarkosyl and 1× high phosphate buffer (0.5 M NaCl, 0.1 M Na₂HPO₄, 5 mM EDTA, pH 7) at 55 C for two hr. The probe, diluted in 15 ml fresh hybridization solution, is then added to the membrane. The membrane is hybridized with the probe at 55 C. for 16 hr. Following hybridization, the membrane is washed for 15 min at 25 C. in 1 mM Tris (pH 8.0), 1% Sarkosyl, and four times for 15 min each at 25 C. in 1 mM Tris (pH 8.0). To detect hybridization complexes, XOMAT-AR film (Eastman Kodak, Rochester N.Y.) is exposed to the membrane overnight at −70 C., developed, and examined.

XVIII Further Characterization of Differentially Expressed cDNAs and Proteins

Sequences were blasted against the LIFESEQ Gold 5.1 database (Incyte Genomics) and an Incyte nucleic acid sequence and its sequence variants were chosen for each clone. The nucleic acid sequence and variant sequences were blasted against GenBank database to acquire annotation. The cDNAs were translated into amino acid sequences which were blasted against the Genpept and other protein databases to acquire annotation and characterization, i.e., structural motifs.

Percent sequence identity can be determined electronically for two or more amino acid or nucleic acid sequences using the MEGALIGN program, a component of LASERGENE software (DNASTAR). The percent identity between two amino acid sequences is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage identity.

Sequences with conserved protein motifs may be searched using the BLOCKS search program. This program analyses sequence information contained in the Swiss-Prot and PROSITE databases and is useful for determining the classification of uncharacterized proteins translated from genomic or cDNA sequences (Bairoch et al.(supra); Attwood et al. (supra). PROSITE database is a useful source for identifying functional or structural domains that are not detected using motifs due to extreme sequence divergence. Using weight matrices, these domains are calibrated against the SWISS-PROT database to obtain a measure of the chance distribution of the matches.

The PRINTS database can be searched using the BLIMPS search program to obtain protein family “fingerprints”. The PRINTS database complements the PROSITE database by exploiting groups of conserved motifs within sequence alignments to build characteristic signatures of different protein families. For both BLOCKS and PRINTS analyses, the cutoff scores for local similarity were: >1300=strong, 1000-1300=suggestive; for global similarity were: p<exp−3; and for strength (degree of correlation) were: >1300=strong, 1000-1300=weak.

Rat cDNAs were identified by the method as described for human cDNAs using clones from rat cDNA libraries.

XIX Expression of the Encoded Protein

Expression and purification of a protein encoded by a cDNA of the invention is achieved using bacterial or virus-based expression systems. For expression in bacteria, cDNA is subcloned into a vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21(DE3). Antibiotic resistant bacteria express the protein upon induction with IPTG. Expression in eukaryotic cells is achieved by infecting Spodoptera frugiperda (Sf9) insect cells with recombinant baculovirus, Autographica californica nuclear polyhedrosis virus. The polyhedrin gene of baculovirus is replaced with the cDNA by either homologous recombination or bacterial-mediated transposition involving transfer plasmid intermediates. Viral infectivity is maintained and the strong polyhedrin promoter drives high levels of transcription.

For ease of purification, the protein is synthesized as a fusion protein with glutathione-S-transferase (GST; APB) or a similar alternative such as FLAG. The fusion protein is purified on immobilized glutathione under conditions that maintain protein activity and antigenicity. After purification, the GST moiety is proteolytically cleaved from the protein with thrombin. A fusion protein with FLAG, an 8-amino acid peptide, is purified using commercially available monoclonal and polyclonal anti-FLAG antibodies (Eastman Kodak, Rochester N.Y.).

XX Production of Specific Antibodies

A denatured protein from a reverse phase HPLC separation is obtained in quantities up to 75 mg. This denatured protein is used to immunize mice or rabbits following standard protocols. About 100 μg is used to immunize a mouse, while up to 1 mg is used to immunize a rabbit. The denatured protein is radioiodinated and incubated with murine B-cell hybridomas to screen for monoclonal antibodies. About 20 mg of protein is sufficient for labeling and screening several thousand clones.

In another approach, the amino acid sequence translated from a cDNA of the invention is analyzed using PROTEAN software (DNASTAR) to determine regions of high antigenicity, essentially antigenically-effective epitopes of the protein. The optimal sequences for immunization are usually at the C-terminus, the N-terminus, and those intervening, hydrophilic regions of the protein that are likely to be exposed to the external environment when the protein is in its natural conformation. Typically, oligopeptides about 15 residues in length are synthesized using an ABI 431 peptide synthesizer (ABI) using Fmoc-chemistry and then coupled to keyhole limpet hemocyanin (KLH; Sigma Aldrich) by reaction with M-maleimidobenzoyl-N-hydroxysuccinimide ester. If necessary, a cysteine may be introduced at the N-terminus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated protein to identify those fusions producing a monoclonal antibody specific for the protein. In a typical protocol, wells of 96 well plates (FAST, Becton-Dickinson, Palo Alto Calif.) are coated with affinity-purified, specific rabbit-anti-mouse (or suitable anti-species Ig) antibodies at 10 mg/ml. The coated wells are blocked with 1% BSA and washed and exposed to supernatants from hybridomas. After incubation, the wells are exposed to radiolabeled protein at 1 mg/ml. Clones producing antibodies bind a quantity of labeled protein that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on protein A (APB). Monoclonal antibodies with affinities of at least 10⁸ M⁻¹, preferably 10⁹ to 10¹⁰ M⁻¹ or stronger, are made by procedures well known in the art.

XXI Purification of Naturally Occurring Protein Using Specific Antibodies

Naturally occurring or recombinant protein is substantially purified by immunoaffinity chromatography using antibodies specific for the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XXII Screening Molecules for Specific Binding with the cDNA or Protein

The cDNA or fragments thereof and the protein or portions thereof are labeled with ³²P-dCTP, Cy3-dCTP, Cy5-dCTP (APB), or BIODIPY or FITC (Molecular Probes), respectively. Candidate molecules or compounds previously arranged on a substrate are incubated in the presence of labeled nucleic or amino acid. After incubation under conditions for either a cDNA or a protein, the substrate is washed, and any position on the substrate retaining label, which indicates specific binding or complex formation, is assayed. The binding molecule is identified by its arrayed position on the substrate. Data obtained using different concentrations of the nucleic acid or protein are used to calculate affinity between the labeled nucleic acid or protein and the bound molecule. High throughput screening using very small assay volumes and very small amounts of test compound is fully described in Burbaum et al. U.S. Pat. No. 5,876,946.

All patents and publications mentioned in the specification are incorporated herein by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| SEQ | | | | | |
|---|---|---|---|---|---|
| ID | | Maximum | | | Representative |
| NO | Clone ID | Abundance | Strain | Sex | Treatment |
| 1 | 700066666 | 6 | HW | | APAP |
| 2 | 700138330 | 4 | HW | | APAP |
| 3 | 700138340 | 3 | HW | | APAP |
| 4 | 700230507 | 3 | HW | | APAP |
| 5 | 700275105 | 3 | HW | | APAP |
| 6 | 700062791 | 4 | HW | | BP |
| 7 | 700250279 | 6 | HW | | BP |
| 8 | 700312413 | 4 | HW | | BP |
| 9 | 700308908 | 6 | HW | | BP, CLO |
| 10 | 700139239 | 4 | HW | M | CLO |
| 11 | 700131403 | 4 | HW | | CLO |
| | | | | | ANIT, 4-AAF, |
| 12 | 700062506 | 3 | HW | F | Hydra |
| | | | | | Feno, 4-AAF, |
| 13 | 700135749 | 4 | HW | F | Hydra |
| 14 | 700330824 | 3 | HW | F | ANIT, CCI4, 4-AAF |
| 15 | 700024728 | 3 | HW | | ANIT, 4-AAF |
| 16 | 700024834 | 3 | HW | | ANIT, CCI4, 4-AAF |
| 17 | 700059927 | 4 | HW | | ANIT, Hydra |
| 18 | 700060675 | 4 | HW | | ANIT, CCI4 |
| 19 | 700062240 | 5 | HW | | ANIT, CCI4, 4-AAF |
| 20 | 700063569 | 3 | HW | | Feno, ANIT |
| 21 | 700064131 | 3 | HW | | ANIT, 4-AAF |
| 22 | 700065081 | 3 | HW | | 4-AAF |
| 23 | 700067778 | 3 | HW | | ANIT, CCI4, Hydra |
| 24 | 700123633 | 3 | HW | | ANIT, CCI4, Hydra |
| 25 | 700127949 | 3 | HW | | CCI4, 4-AAF |
| 26 | 700132557 | 7 | HW | | CCI4, 4-AAF |
| 27 | 700135733 | 4 | HW | | CCI4, 4-AAF |
| | | | | | CCI4, 4-AAF, |
| 28 | 700135850 | 3 | HW | | Hydra |
| 29 | 700137416 | 4 | HW | | ANIT, CCI4, |
| 30 | 700140375 | 3 | HW | | Feno, CCI4, 4-AAF |
| | | | | | Feno, ANIT, CCI4, |
| 31 | 700140450 | 8 | HW | | 4-AAF |
| 32 | 700144406 | 4 | HW | | ANIT, CCI4, Hydra |
| 33 | 700175249 | 7 | HW | | ANIT, CCI4, Hydra |
| 34 | 700177158 | 3 | HW | | ANIT, CCI4, 4-AAF |
| 35 | 700179766 | 3 | HW | | Feno, ANIT |
| 36 | 700181614 | 3 | HW | | ANIT, CCI4 |
| 37 | 700182016 | 3 | HW | | Feno, CCI4, 4-AAF |
| | | | | | Feno, ANIT, CCI4, |
| 38 | 700185871 | 3 | HW | | 4-AAF |
| 39 | 700198357 | 3 | HW | | Feno, ANIT, CCI4 |
| 40 | 700230123 | 4 | HW | | Feno, ANIT, CCI4 |
| 41 | 700248367 | 3 | HW | | ANIT, CCI4, 4-AAF |
| 42 | 700250877 | 3 | HW | | Feno, CCI4 |
| 43 | 700253694 | 3 | HW | | CCI4, 4-AAF |
| 44 | 700303850 | 3 | HW | | CCI4, 4-AAF |
| | | | | | Feno, ANIT, CCI4, |
| 45 | 700305380 | 4 | HW | | 4-AAF |
| 46 | 700313077 | 3 | HW | | CCI4 |
| 47 | 700361225 | 3 | HW | | CCI4 |
| 48 | 700363174 | 3 | HW | | ANIT, CCI4, 4-AAF |
| 49 | 700478138 | 5 | HW | | Feno, ANIT, CCI4 |
| 50 | 700480077 | 3 | HW | | ANIT, CCI4, 4-AAF |
| 51 | 700483222 | 3 | HW | | ANIT, 4-AAF |
| 52 | 700491990 | 3 | HW | | CCI4 |
| | | | | | Feno, ANIT, CCI4, |
| 53 | 700818852 | 5 | HW | | 4-AAF |
| | | | | | Feno, ANIT, CCI4, |
| 54 | 700937735 | 8 | HW | | 4-AAF |
| 55 | 701197728 | 3 | HW | | Feno, ANIT |
| 56 | 701257766 | 3 | HW | | ANIT, CCI4 |
| 57 | 701341861 | 3 | HW | | ANIT, 4-AAF |
| 58 | 701574951 | 3 | HW | | CCI4, 4-AAF |
| 59 | 701578278 | 3 | HW | | CCI4 |
| 60 | 701597659 | 3 | HW | | Feno, ANIT, CCI4 |
| | | | | | ANIT, CCI4, |
| 61 | 701637512 | 4 | HW | | 4-AAF, Hydra |
| 62 | 700139271 | 8 | SD | M | APAP |
| 63 | 700141770 | 3 | SD | M | APAP |
| 64 | 700142213 | 4 | SD | M | APAP |
| 65 | 700128536 | 3 | SD | | APAP |
| 66 | 700187893 | 3 | SD | | APAP |
| 67 | 700285351 | 3 | SD | | APAP |
| 68 | 700480580 | 3 | SD | | APAP |
| 69 | 701255247 | 4 | SD | | APAP |
| 70 | 701471433 | 3 | SD | | APAP |
| 71 | 701478494 | 3 | SD | | APAP |
| 72 | 701483549 | 4 | SD | | APAP |
| 73 | 701942330 | 3 | SD | | APAP |
| 74 | 700137978 | 7 | SD | F | BP |
| 75 | 701709967 | 4 | SD | F | BP |
| 76 | 700059750 | 3 | SD | | BP |
| 77 | 700123903 | 3 | SD | | BP |
| 78 | 700135554 | 3 | SD | | BP |
| 79 | 700175783 | 4 | SD | | BP |
| 80 | 700176825 | 4 | SD | | BP |
| 81 | 700177142 | 4 | SD | | BP |
| 82 | 700185570 | 3 | SD | | BP |
| 83 | 700244879 | 3 | SD | | BP |
| 84 | 700246085 | 4 | SD | | BP |
| 85 | 700302152 | 10 | SD | | BP |
| 86 | 700305538 | 13 | SD | | BP |
| 87 | 700307329 | 3 | SD | | BP |
| 88 | 700323326 | 4 | SD | | BP |
| 89 | 700328990 | 4 | SD | | BP |
| 90 | 700330391 | 3 | SD | | BP |
| 91 | 700363445 | 6 | SD | | BP |
| 92 | 700480903 | 3 | SD | | BP |
| 93 | 700482908 | 3 | SD | | BP |
| 94 | 700935261 | 3 | SD | | BP |
| 95 | 700939101 | 3 | SD | | BP |
| 96 | 700950626 | 3 | SD | | BP |
| 97 | 701316308 | 3 | SD | | BP |
| 98 | 701316810 | 3 | SD | | BP |
| 99 | 701327688 | 3 | SD | | BP |
| 100 | 701337820 | 3 | SD | | BP |
| 101 | 701432276 | 4 | SD | | BP |
| 102 | 701432317 | 3 | SD | | BP |
| 103 | 701472012 | 3 | SD | | BP |
| 104 | 701472036 | 3 | SD | | BP |
| 105 | 701517108 | 3 | SD | | BP |
| 106 | 701710808 | 4 | SD | | BP |
| 107 | 701241446 | 3 | SD | | BP, APAP |
| 108 | 701711993 | 5 | SD | | BP, APAP |
| 109 | 700250903 | 11 | SD | M | BP, CLO |
| 110 | 700192259 | 5 | SD | | BP, CLO |
| 111 | 700269330 | 4 | SD | | BP, CLO |
| 112 | 701317696 | 5 | SD | | BP, CLO |
| 113 | 700230724 | 4 | SD | F | CLO |
| 114 | 700307025 | 4 | SD | F | CLO |
| 115 | 700361295 | 3 | SD | F | CLO |
| 116 | 700420930 | 3 | SD | F | CLO |
| 117 | 700526706 | 3 | SD | F | CLO |
| 118 | 700635732 | 3 | SD | F | CLO |
| 119 | 700824178 | 3 | SD | F | CLO |
| 120 | 701396706 | 4 | SD | F | CLO |
| 121 | 701475987 | 3 | SD | F | CLO |
| 122 | 701621131 | 3 | SD | F | CLO |
| 123 | 701879551 | 3 | SD | F | CLO |
| 124 | 701883446 | 3 | SD | F | CLO |
| 125 | 701883859 | 3 | SD | F | CLO |
| 126 | 700122019 | 4 | SD | M | CLO |
| 127 | 700276321 | 4 | SD | M | CLO |
| 128 | 700323126 | 3 | SD | M | CLO |
| 129 | 700324604 | 3 | SD | M | CLO |
| 130 | 700363120 | 3 | SD | M | CLO |
| 131 | 700364810 | 3 | SD | M | CLO |
| 132 | 700510701 | 4 | SD | M | CLO |
| 133 | 700810877 | 5 | SD | M | CLO |
| 134 | 700928985 | 6 | SD | M | CLO |
| 135 | 700931410 | 4 | SD | M | CLO |
| 136 | 701093657 | 4 | SD | M | CLO |
| 137 | 701259518 | 3 | SD | M | CLO |
| 138 | 701264516 | 4 | SD | M | CLO |
| 139 | 701341715 | 4 | SD | M | CLO |
| 140 | 701434939 | 4 | SD | M | CLO |
| 141 | 701460429 | 3 | SD | M | CLO |
| 142 | 701463285 | 4 | SD | M | CLO |
| 143 | 701605992 | 4 | SD | M | CLO |
| 144 | 701737185 | 3 | SD | M | CLO |
| 145 | 701737221 | 4 | SD | M | CLO |
| 146 | 701920170 | 3 | SD | M | CLO |
| 147 | 701922204 | 3 | SD | M | CLO |
| 148 | 701922375 | 4 | SD | M | CLO |
| 149 | 701922439 | 4 | SD | M | CLO |
| 150 | 701922513 | 4 | SD | M | CLO |
| 151 | 701922576 | 4 | SD | M | CLO |
| 152 | 701922583 | 3 | SD | M | CLO |
| 153 | 701922711 | 3 | SD | M | CLO |
| 154 | 701922744 | 4 | SD | M | CLO |
| 155 | 701922971 | 3 | SD | M | CLO |
| 156 | 701923019 | 5 | SD | M | CLO |
| 157 | 701923022 | 3 | SD | M | CLO |
| 158 | 701923218 | 4 | SD | M | CLO |
| 159 | 701923240 | 4 | SD | M | CLO |
| 160 | 701923241 | 3 | SD | M | CLO |
| 161 | 701923330 | 3 | SD | M | CLO |
| 162 | 701925484 | 4 | SD | M | CLO |
| 163 | 700025278 | 4 | SD | | CLO |
| 164 | 700122008 | 6 | SD | | CLO |
| 165 | 700136662 | 3 | SD | | CLO |
| 166 | 700144043 | 4 | SD | | CLO |
| 167 | 700177423 | 3 | SD | | CLO |
| 168 | 700247621 | 4 | SD | | CLO |
| 169 | 700251379 | 3 | SD | | CLO |
| 170 | 700272988 | 5 | SD | | CLO |
| 171 | 700306859 | 3 | SD | | CLO |
| 172 | 700307013 | 3 | SD | | CLO |
| 173 | 700323707 | 4 | SD | | CLO |
| 174 | 700513589 | 4 | SD | | CLO |
| 175 | 700538641 | 3 | SD | | CLO |
| 176 | 700592657 | 3 | SD | | CLO |
| 177 | 701190918 | 3 | SD | | CLO |
| 178 | 701313456 | 3 | SD | | CLO |
| 179 | 701397319 | 3 | SD | | CLO |
| 180 | 701429135 | 6 | SD | | CLO |
| 181 | 701429654 | 3 | SD | | CLO |
| 182 | 701458841 | 3 | SD | | CLO |
| 183 | 701470083 | 3 | SD | | CLO |
| 184 | 701562608 | 3 | SD | | CLO |
| 185 | 701884534 | 5 | SD | | CLO |
| 186 | 700068662 | 6 | SD | | CLO, BP |
| 187 | 700933309 | 5 | SD | | CLO, BP |
| 188 | 700024288 | 2 | SD | F | CLO, BP |
| 189 | 700064545 | 3 | SD | F | CLO |
| 190 | 700066933 | 2 | SD | F | CLO, BP |
| 191 | 700146747 | 3 | SD | F | CLO, BP |
| 192 | 700589657 | 3 | SD | F | CLO |
| 193 | 700858043 | 1 | SD | F | CLO, BP, APAP |
| 194 | 700060595 | 3 | SD | M | CLO |
| 195 | 700061212 | 3 | SD | M | CLO |
| 196 | 700062747 | 2 | SD | M | CLO, BP, APAP |
| 197 | 700124248 | 3 | SD | M | CLO, BP |
| 198 | 700252787 | 3 | SD | M | CLO |
| 199 | 700272719 | 3 | SD | M | CLO |
| 200 | 700282789 | 3 | SD | M | CLO, BP, APAP |
| 201 | 700062503 | 3 | SD | | CLO, BP |
| 202 | 700490891 | 4 | SD | | BP, APAP |

TABLE 2

| | | | | Human | | |
|---|---|---|---|---|---|---|
| | | | | Template | | |
| | Rat Template | Human Template | Human Template | BLAST Hit | | |
| SEQ ID NO | SEQ ID NO | Number | SEQ ID NO | (Genbank ID) | Hit Description | E value |
| 1 | 210 | 232589.78 | 409 | g4929559 | CGI-45 protein | 1e−24 |
| 2 | 219 | | | | | |
| 3 | 261 | | | | | |
| 4 | 296 | 197046.4 | 467 | g6841323 | Human HSPC337 mRNA, partial cds. | 0 |
| 5 | 252 | 234758.1 | 431 | g6457341 | Human E2IG4 (E2IG4) mRNA, | 0 |
| | | | | | complete cds. | |
| 6 | 231 | 1327511.1 | 416 | g7294014 | CG7725 gene product | 4e−29 |
| 7 | 205 | 1383009.65 | 402 | g6979641 | Human alpha gene sequence. | 0 |
| 8 | 204 | 348148.41 | 401 | g32451 | Human pHS1-2 mRNA with ORF | 0 |
| | | | | | homologous to membrane receptor | |
| | | | | | proteins. | |
| 9 | 264 | | | | | |
| 10 | 246 | | | | | |
| 11 | 280 | 1035717.1 | 456 | g6807782 | Human mRNA; cDNA DKFZp434P086 | 0 |
| | | | | | (from clone DKFZp434P086); | |
| | | | | | partial cds. | |
| 12 | 232 | 380601.63 | 418 | g183891 | Human high density lipoprotein | 0 |
| | | | | | binding protein (HBP) mRNA, | |
| | | | | | complete cds. | |
| 13 | 248 | 1099747.1 | 428 | g5817098 | Human mRNA; cDNA DKFZp566D211 | 8e−62 |
| | | | | | (from clone DKFZp566D211). | |
| 14 | 222 | 978146.2 | 412 | g2865252 | unknown | 6e−15 |
| 15 | 297 | 1311223.1 | 468 | g5138919 | Human PTD014 mRNA, complete cds. | 0 |
| 16 | 263 | 1001899.1 | 441 | | Incyte Unique | |
| 17 | 268 | 1247195.1 | 446 | g6690235 | Human clone HQ0569. | 0 |
| 18 | 284 | | | | | |
| 19 | 287 | 232376.7 | 459 | g7023163 | unnamed protein product | 2e−63 |
| 20 | 227 | | | | | |
| 21 | 377 | | | | | |
| 22 | 383 | 411426.42 | 513 | g338046 | Human SF2p33 mRNA, complete cds. | 0 |
| 23 | 247 | 234677.1 | 427 | g5052586 | BcDNA.GH08385 | 4e−22 |
| 24 | 361 | 236253.20 | 503 | g6808164 | Human mRNA; cDNA DKFZp761A052 | 0 |
| | | | | | (from clone DKFZp761A052). | |
| 25 | 249 | | | | | |
| 26 | 399 | | | | | |
| 27 | 226 | 234482.3 | 414 | g6807766 | Human mRNA; cDNA DKFZp434E146 | 0 |
| | | | | | (from clone DKFZp434E146). | |
| 28 | 336 | 337394.18 | 497 | g7023484 | Human cDNA FLJ11059 fis, clone | 0 |
| | | | | | PLACE1004740. | |
| 29 | 223 | 347045.1 | 413 | g7020630 | Human cDNA FLJ20493 fis, clone | 0 |
| | | | | | KAT08512. | |
| 30 | 238 | | | | | |
| 31 | 324 | 978075.1 | 489 | g7300427 | CG7709 gene product | 1e−05 |
| 32 | 207 | 366288.5 | 406 | g7688684 | Human AD-015 protein mRNA, | 0 |
| | | | | | complete cds. | |
| 33 | 304 | 018653.18 | 474 | | Incyte Unique | |
| 34 | 367 | 235106.13 | 506 | g7020192 | Human cDNA FLJ20234 fis, clone | 0 |
| | | | | | COLF5673. | |
| 35 | 303 | 038495.8 | 473 | g7023545 | Human cDNA FLJ11095 fis, clone | 0 |
| | | | | | PLACE1005374. | |
| 36 | 300 | 474711.6 | 471 | g7959200 | Human mRNA for KIAA1470 protein, | 0 |
| | | | | | partial cds. | |
| 37 | 239 | 071944.2 | 422 | g7106795 | Human HSPC203 mRNA, complete | 0 |
| | | | | | cds. | |
| 38 | 269 | 474736.11 | 447 | g6457337 | Human E2IG1 (E2IG1) mRNA, | 0 |
| | | | | | complete cds. | |
| 39 | 256 | 480324.16 | 436 | g7208833 | hypothetical protein | 0 |
| 40 | 313 | 246290.8 | 481 | g7022551 | unnamed protein product | 7e−38 |
| 41 | 250 | 216242.2 | 429 | g5565686 | Human topoisomerase-related | 0 |
| | | | | | function protein (TRF4-1) mRNA, | |
| | | | | | partial cds. | |
| 42 | 234 | 238854.22 | 420 | g190258 | Human neuron-specific protein | 0 |
| | | | | | gene, last exon, clone D4S234. | |
| 43 | 290 | 1166953.1 | 464 | g7959742 | Human PRO1068 mRNA, complete | 0 |
| | | | | | cds. | |
| 44 | 320 | | | | | |
| 45 | 360 | 333057.2 | 502 | g179717 | Human complement protein C8 | 0 |
| | | | | | alpha subunit mRNA, complete | |
| | | | | | cds. | |
| 46 | 330 | 200068.22 | 494 | g5114044 | Human N-terminal | 0 |
| | | | | | acetyltransferase complex ard1 | |
| | | | | | subunit mRNA, complete cds. | |
| 47 | 278 | 020434.12 | 454 | g7023192 | Human cDNA FLJ10879 fis, clone | 0 |
| | | | | | NT2RP4001896, weakly similar to | |
| | | | | | VEGETATIBLE INCOMPATIBILITY | |
| | | | | | PROTEIN HET-E-1. | |
| 48 | 288 | 1383610.2 | 460 | g3877100 | predicted using | 3e−44 |
| | | | | | Genefinder˜Similarity to Yeast | |
| | | | | | mitochondrial ribosomal protein | |
| | | | | | S5 (SW: RT05_YEAST) | |
| 49 | 381 | | | | | |
| 50 | 283 | | | | | |
| 51 | 334 | 334177.1 | 496 | g7294197 | CG13076 gene product | 3e−88 |
| 52 | 309 | | | | | |
| 53 | 261 | | | | | |
| 54 | 331 | | | | | |
| 55 | 305 | 097341.1 | 475 | g3873789 | predicted using Genefinder | 1e−23 |
| 56 | 257 | 041856.14 | 437 | g7296234 | CG3645 gene product | 0 |
| 57 | 376 | | | | | |
| 58 | 355 | | | | | |
| 59 | 274 | 481118.12 | 451 | g6434473 | predicted using Genefinder; | 2e−10 |
| | | | | | preliminary prediction | |
| 60 | 353 | | | | | |
| 61 | 285 | | | | | |
| 62 | 372 | | | | | |
| 63 | 322 | 1397900.1 | 487 | g5821151 | RNA binding protein | 0.001 |
| 64 | 331 | | | | | |
| 65 | 363 | 246362.2 | 504 | g6841259 | Human HSPC305 mRNA, partial cds. | 0 |
| 66 | 262 | | | | | |
| 67 | 362 | | | | | |
| 68 | 251 | 174274.1 | 430 | | Public Unique | |
| 69 | 293 | | | | | |
| 70 | 225 | | | | | |
| 71 | 224 | | | | | |
| 72 | 208 | 1245800.1 | 407 | g180947 | Human carboxylesterase mRNA, | 0 |
| | | | | | complete cds. | |
| 73 | 335 | | | | | |
| 74 | 344 | | | | | |
| 75 | 352 | | | | | |
| 76 | 221 | | | | | |
| 77 | 323 | 903849.1 | 488 | g7020507 | Human cDNA FLJ20420 fis, clone | 0 |
| | | | | | KAT02462. | |
| 78 | 243 | 991612.1 | 425 | g6969165 | dJ475N16.3 (novel protein | 0 |
| | | | | | similar to RPL7A (60S ribosomal | |
| | | | | | protein L7A)) | |
| 79 | 282 | 1002701.1 | 458 | g7020763 | Human cDNA FLJ20568 fis, clone | 1e−58 |
| | | | | | REC00775. | |
| 80 | 255 | 1042482.1 | 435 | g189786 | erythrocyte p55 | 5e−79 |
| 81 | 326 | 348080.7 | 491 | g5360100 | Human NY-REN-25 antigen mRNA, | 0 |
| | | | | | partial cds. | |
| 82 | 253 | 407217.1 | 432 | | Incyte Unique | |
| 83 | 382 | 1019222.1 | 512 | g3342730 | R31341_1 | 4e−21 |
| 84 | 299 | 335705.2 | 470 | g1136435 | Human mRNA for KIAA0188 gene, | 0 |
| | | | | | partial cds. | |
| 85 | 384 | | | | | |
| 86 | 384 | | | | | |
| 87 | 270 | 1098449.1 | 448 | g6457339 | Human E2IG3 (E2IG3) mRNA, | 4e−34 |
| | | | | | complete cds. | |
| 88 | 369 | | | | | |
| 89 | 215 | | | | | |
| 90 | 266 | 230889.9 | 443 | g5817126 | Human mRNA; cDNA DKFZp586P1622 | 0 |
| | | | | | (from clone DKFZp586P1622). | |
| 91 | 318 | 985408.10 | 484 | g7296240 | CG11490 gene product | 5e−81 |
| 92 | 307 | 203528.1 | 477 | | Incyte Unique | |
| 93 | 271 | | | | | |
| 94 | 378 | 406695.4 | 509 | g6634013 | KIAA0310 protein | 2e−52 |
| 95 | 245 | | | | | |
| 96 | 292 | | | | | |
| 97 | 356 | | | | | |
| 98 | 302 | | | | | |
| 99 | 298 | 205241.6 | 469 | g2772914 | precollagen D | 5e−31 |
| 100 | 240 | 332413.6 | 423 | g6453539 | Human mRNA; cDNA DKFZp434D0428 | 0 |
| | | | | | (from clone DKFZp434D0428); | |
| | | | | | partial cds. | |
| 101 | 217 | | | | | |
| 102 | 348 | | | | | |
| 103 | 213 | | | | | |
| 104 | 354 | 206504.1 | 501 | | Incyte Unique | |
| 105 | 214 | | | | | |
| 106 | 350 | | | | | |
| 107 | 337 | | | | | |
| 108 | 351 | | | | | |
| 109 | 209 | 196677.1 | 408 | g7022989 | Human cDNA FLJ10761 fis, clone | 0 |
| | | | | | NT2RP3004669, weakly similar to | |
| | | | | | ETHANOLAMINE KINASE (EC | |
| | | | | | 2.7.1.82). | |
| 110 | 275 | | | | | |
| 111 | 277 | 1135179.1 | 453 | g6002488 | hypothetical protein | 2e−26 |
| 112 | 368 | | | | | |
| 113 | 242 | | | | | |
| 114 | 316 | | | | | |
| 115 | 317 | 216452.26 | 483 | g7300920 | CG17141 gene product | 1e−65 |
| 116 | 375 | | | | | |
| 117 | 228 | 027980.2 | 415 | g7243746 | Human sir2-related protein type | 0 |
| | | | | | 7 (SIRT7) mRNA, complete cds. | |
| 118 | 321 | 238273.6 | 486 | g6434875 | Human CDK4-binding protein | 0 |
| | | | | | p34SEI1 (SEI1) mRNA, complete | |
| | | | | | cds. | |
| 119 | 244 | 331609.14 | 426 | g7301549 | CG5880 gene product | 1e−70 |
| 120 | 235 | | | | | |
| 121 | 218 | | | | | |
| 122 | 349 | | | | | |
| 123 | 357 | | | | | |
| 124 | 397 | | | | | |
| 125 | 345 | 326679.1 | 499 | | Incyte Unique | |
| 126 | 312 | 331451.18 | 480 | g5262615 | hypothetical protein | 2e−22 |
| 127 | 366 | | | | | |
| 128 | 281 | 1346179.1 | 457 | | Incyte Unique | |
| 129 | 380 | 246935.4 | 511 | g2292986 | cyclic nucleotide-gated channel | 2e−08 |
| | | | | | beta subunit | |
| 130 | 315 | 174240.1 | 482 | g6453490 | Human mRNA; cDNA DKFZp434A2417 | 1e−27 |
| | | | | | (from clone DKFZp434A2417); | |
| | | | | | partial cds. | |
| 131 | 265 | 198345.3 | 442 | g4973018 | Human APMCF1 (APMCF1) mRNA, | 0 |
| | | | | | complete cds. | |
| 132 | 316 | | | | | |
| 133 | 260 | | | | | |
| 134 | 289 | 410014.15 | 462 | g2605967 | 24 | 2e−13 |
| 135 | 333 | 199581.5 | 495 | g5441951 | Human peroxisomal membrane | 0 |
| | | | | | protein PMP 24 mRNA, complete | |
| | | | | | cds. | |
| 136 | 365 | | | | | |
| 137 | 258 | 252542.6 | 438 | g178120 | Human class II alcohol | 0 |
| | | | | | dehydrogenase (ADH4) pi subunit | |
| | | | | | mRNA, complete cds. | |
| 138 | 286 | | | | | |
| 139 | 343 | | | | | |
| 140 | 212 | | | | | |
| 141 | 329 | 992317.12 | 493 | g6690017 | NTR | 8e−12 |
| 142 | 342 | | | | | |
| 143 | 306 | 242821.5 | 476 | g7021948 | Human cDNA FLJ10099 fis, clone | 0 |
| | | | | | HEMBA1002462. | |
| 144 | 392 | | | | | |
| 145 | 385 | | | | | |
| 146 | 338 | | | | | |
| 147 | 339 | | | | | |
| 148 | 386 | | | | | |
| 149 | 387 | | | | | |
| 150 | 393 | | | | | |
| 151 | 394 | | | | | |
| 152 | 396 | | | | | |
| 153 | 390 | | | | | |
| 154 | 391 | | | | | |
| 155 | 371 | | | | | |
| 156 | 389 | | | | | |
| 157 | 388 | | | | | |
| 158 | 276 | 149914.32 | 452 | g7022963 | Human cDNA FLJ10744 fis, clone | 0 |
| | | | | | NT2RP3001646, weakly similar to | |
| | | | | | WD-40 REPEAT PROTEIN MSI2. | |
| 159 | 340 | 1099669.1 | 498 | g7018544 | Human mRNA; cDNA DKFZp434F1312 | 0 |
| | | | | | (from clone DKFZp434F1312); | |
| | | | | | partial cds. | |
| 160 | 395 | | | | | |
| 161 | 398 | | | | | |
| 162 | 341 | | | | | |
| 163 | 301 | 903554.2 | 472 | g6164677 | Human | 0 |
| | | | | | methylmalonate-semialdehyde | |
| | | | | | dehydrogenase (MMSDH) mRNA, | |
| | | | | | complete cds. | |
| 164 | 308 | | | | | |
| 165 | 294 | 244999.5 | 465 | | Incyte Unique | |
| 166 | 311 | 475017.1 | 479 | g7291808 | CG11414 gene product | 2e−20 |
| 167 | 237 | | | | | |
| 168 | 310 | 229079.16 | 478 | g3242705 | unknown protein | 0 |
| 169 | 328 | | | | | |
| 170 | 359 | | | | | |
| 171 | 314 | | | | | |
| 172 | 325 | 1340709.1 | 490 | g5912188 | Human mRNA; cDNA DKFZp564B1264 | 6e−11 |
| | | | | | (from clone DKFZp564B1264). | |
| 173 | 279 | 232928.5 | 455 | g7023105 | Human cDNA FLJ10828 fis, clone | 0 |
| | | | | | NT2RP4001122, weakly similar to | |
| | | | | | TIPD PROTEIN. | |
| 174 | 327 | 1382922.15 | 492 | g337515 | Human ribosomal protein S6 mRNA, | 0 |
| | | | | | complete cds. | |
| 175 | 254 | 246916.11 | 433 | g7106865 | Human HSPC238 mRNA, complete | 0 |
| | | | | | cds. | |
| 176 | 203 | 235885.5 | 400 | g3298561 | Human zinc-finger helicase | 0 |
| | | | | | (hZFH) mRNA, complete cds. | |
| 177 | 273 | 228579.1 | 450 | | Incyte Unique | |
| 178 | 370 | | | | | |
| 179 | 230 | | | | | |
| 180 | 358 | | | | | |
| 181 | 206 | 369213.39 | 405 | g35824 | Human mRNA for pregnancy zone | 0 |
| | | | | | protein. | |
| 182 | 332 | | | | | |
| 183 | 229 | | | | | |
| 184 | 347 | 474680.27 | 500 | g3947689 | Human mRNA for sec24 protein | 0 |
| | | | | | (Sec24B isoform). | |
| 185 | 291 | | | | | |
| 186 | 211 | | | | | |
| 187 | 322 | 1397900.1 | 487 | g5821151 | RNA binding protein | 0.001 |
| 188 | 216 | 308057.1 | 410 | | Public Unique | |
| 189 | 236 | 967709.1 | 421 | g7303346 | CG17059 gene product | 1e−08 |
| 190 | 220 | 1137592.1 | 411 | | Incyte Unique | |
| 191 | 319 | 413491.14 | 485 | g7023393 | Human cDNA FLJ11000 fis, clone | 0 |
| | | | | | PLACE1002794. | |
| 192 | 295 | 239501.1 | 466 | g7021134 | Human cDNA FLJ20815 fis, clone | 0 |
| | | | | | ADSE01038, highly similar to | |
| | | | | | AJ007398 Human mRNA for PBK1 | |
| | | | | | protein. | |
| 193 | 259 | 233720.7 | 439 | g7022206 | Human cDNA FLJ10276 fis, clone | 0 |
| | | | | | HEMBB1001182. | |
| 194 | 346 | | | | | |
| 195 | 379 | 892236.1 | 510 | | Incyte Unique | |
| 196 | 233 | 235169.27 | 419 | g6841281 | Human HSPC316 mRNA, partial cds. | 0 |
| 197 | 364 | 198141.3 | 505 | g7020508 | unnamed protein product | 3e−27 |
| 198 | 373 | | | | | |
| 199 | 374 | 404701.2 | 508 | g5020383 | juvenile hormone esterase | 9e−16 |
| | | | | | binding protein | |
| 200 | 241 | 330897.2 | 424 | g1504007 | Human mRNA for KIAA0212 gene, | 0 |
| | | | | | complete cds. | |
| 201 | 267 | 1248547.1 | 445 | g7023268 | Human cDNA FLJ10920 fis, clone | 0 |
| | | | | | OVARC1000384. | |
| 202 | 272 | 427529.10 | 449 | g7332115 | contains similarity to | 2e−23 |
| | | | | | Mycobacterium tuberculosis bpoC | |
| | | | | | (GB: Z95558) | |

TABLE 3

| | Human | |
|---|---|---|
| | Template | |
| SEQ ID NO | ID | Tissue Distribution |
| 490 | 1340709.1 | Cardiovascular System - 100% |
| 433 | 246916.11 | Cardiovascular System - 12% |
| 486 | 238273.6 | Connective Tissue - 10%, Female Genitalia - 10% |
| 425 | 991612.1 | Connective Tissue - 12% |
| 422 | 71944.2 | Connective Tissue - 13%, Cardiovascular System - 12%, |
| | | Embryonic Structures - 11% |
| 419 | 235169.27 | Connective Tissue - 19%, Unclassified/Mixed - 16% |
| 452 | 149914.32 | Connective Tissue - 20%, Embryonic Structures - 15%, |
| | | Urinary Tract - 14% |
| 495 | 199581.5 | Connective Tissue - 39%, Exocrine Glands - 22%, Digestive |
| | | System - 22% |
| 499 | 326679.1 | Digestive System - 100% |
| 478 | 229079.16 | Digestive System - 14%, Pancreas - 14%, Respiratory System - 11% |
| 492 | 1382922.15 | Embryonic Structures - 11% |
| 483 | 216452.26 | Embryonic Structures - 11%, Cardiovascular System - 11%, |
| | | Liver - 11% |
| 474 | 18653.18 | Embryonic Structures - 13%, Stomatognathic System - 11% |
| 420 | 238854.22 | Embryonic Structures - 14%, Sense Organs - 13%, |
| | | Unclassified/Mixed - 11%, Skin - 11% |
| 491 | 348080.7 | Embryonic Structures - 21%, Pancreas - 13%, Skin - 10% |
| 487 | 1397900.1 | Embryonic Structures - 21%, Unclassified/Mixed - 13%, |
| | | Digestive System - 11% |
| 496 | 334177.1 | Embryonic Structures - 58%, Liver - 16%, Unclassified/Mixed - 15% |
| 459 | 232376.7 | Endocrine System - 13% |
| 498 | 1099669.1 | Endocrine System - 15%, Male Genitalia - 15%, Nervous |
| | | System - 15% |
| 426 | 331609.14 | Exocrine Glands - 16%, Pancreas - 14%, Musculoskeletal |
| | | System - 10% |
| 461 | 410014.14 | Exocrine Glands - 47%, Musculoskeletal System - 22%, |
| | | Urinary Tract - 12% |
| 449 | 427529.1 | Female Genitalia - 11%, Urinary Tract - 10% |
| 411 | 1137592.1 | Female Genitalia - 67%, Nervous System - 33% |
| 442 | 198345.3 | Germ Cells - 11% |
| 401 | 348148.41 | Germ Cells - 12%, Male Genitalia - 11% |
| 415 | 27980.2 | Germ Cells - 13%, Hemic and Immune System - 11% |
| 479 | 475017.1 | Germ Cells - 14% |
| 504 | 246362.2 | Germ Cells - 14%, Stomatognathic System - 14%, |
| | | Unclassified/Mixed - 13% |
| 477 | 203528.1 | Germ Cells - 16%, Liver - 11%, Cardiovascular System - 10% |
| 427 | 234677.1 | Germ Cells - 18%, Embryonic Structures - 10% |
| 480 | 331451.18 | Germ Cells - 20%, Digestive System - 13%, |
| | | Unclassified/Mixed - 11% |
| 444 | 1307204.1 | Germ Cells - 21%, Pancreas - 12% |
| 460 | 1383610.2 | Germ Cells - 25%, Urinary Tract - 11% |
| 403 | 411384.2 | Germ Cells - 26%, Embryonic Structures - 19%, Male |
| | | Genitalia - 10% |
| 503 | 236253.2 | Germ Cells - 28% |
| 511 | 246935.4 | Germ Cells - 30% |
| 481 | 246290.8 | Germ Cells - 30%, Male Genitalia - 11% |
| 469 | 205241.6 | Germ Cells - 40% |
| 464 | 1166953.1 | Hemic and Immune System - 100% |
| 416 | 1327511.1 | Liver - 100% |
| 428 | 1099747.1 | Liver - 100% |
| 435 | 1042482.1 | Liver - 100% |
| 441 | 1001899.1 | Liver - 100% |
| 448 | 1098449.1 | Liver - 100% |
| 456 | 1035717.1 | Liver - 100% |
| 457 | 1346179.1 | Liver - 100% |
| 458 | 1002701.1 | Liver - 100% |
| 512 | 1019222.1 | Liver - 100% |
| 514 | 1022716.1 | Liver - 100% |
| 446 | 1247195.1 | Liver - 14%, Embryonic Structures - 13% |
| 431 | 234758.1 | Liver - 15%, Exocrine Glands - 10% |
| 489 | 978075.1 | Liver - 16%, Hemic and Immune System - 15%, Endocrine |
| | | System - 11%, Pancreas - 11% |
| 402 | 1383009.65 | Liver - 21%, Female Genitalia - 14%, Embryonic Structures - |
| | | 10%, Nervous System - 10% |
| 404 | 369213.28 | Liver - 23%, Respiratory System - 11% |
| 501 | 206504.1 | Liver - 26%, Unclassified/Mixed - 23%, Hemic and Immune |
| | | System - 20% |
| 405 | 369213.39 | Liver - 30% |
| 475 | 97341.1 | Liver - 35%, Urinary Tract - 27%, Cardiovascular System - |
| | | 15%, Endocrine System - 15% |
| 407 | 1245800.1 | Liver - 38%, Respiratory System - 14% |
| 438 | 252542.6 | Liver - 86% |
| 502 | 333057.2 | Liver - 88% |
| 421 | 967709.1 | Liver - 90%, Hemic and Immune System - 10% |
| 447 | 474736.11 | Male Genitalia - 10% |
| 434 | 246916.15 | Male Genitalia - 40%, Digestive System - 40%, Hemic and |
| | | Immune System - 20% |
| 410 | 308057.1 | Musculoskeletal System - 100% |
| 432 | 407217.1 | Musculoskeletal System - 34%, Stomatognathic System - 29%, |
| | | Liver - 14% |
| 430 | 174274.1 | Nervous System - 100% |
| 482 | 174240.1 | Nervous System - 100% |
| 510 | 892236.1 | Nervous System - 100% |
| 400 | 235885.5 | Nervous System - 11% |
| 414 | 234482.3 | Sense Organs - 11% |
| 468 | 1311223.1 | Sense Organs - 12% |
| 467 | 197046.4 | Sense Organs - 17%, Musculoskeletal System - 13%, Endocrine |
| | | System - 12% |
| 508 | 404701.2 | Sense Organs - 18%, Unclassified/Mixed - 14%, Embryonic |
| | | Structures - 11% |
| 450 | 228579.1 | Sense Organs - 25%, Germ Cells - 13%, Musculoskeletal |
| | | System - 12% |
| 488 | 903849.1 | Skin - 12%, Cardiovascular System - 11% |
| 471 | 474711.6 | Skin - 13%, Germ Cells - 12% |
| 505 | 198141.3 | Skin - 13%, Sense Organs - 12% |
| 424 | 330897.2 | Skin - 15%, Embryonic Structures - 14%, Hemic and Immune |
| | | System - 14% |
| 506 | 235106.13 | Skin - 23%, Digestive System - 17%, Exocrine Glands - 12% |
| 451 | 481118.12 | Stomatognathic System - 11% |
| 497 | 337394.18 | Stomatognathic System - 11% |
| 500 | 474680.27 | Stomatognathic System - 17%, Germ Cells - 11%, Embryonic |
| | | Structures - 10% |
| 423 | 332413.6 | Stomatognathic System - 17%, Unclassified/Mixed - 12% |
| 413 | 347045.1 | Stomatognathic System - 20%, Germ Cells - 17% |
| 509 | 406695.4 | Stomatognathic System - 26%, Digestive System - 10% |
| 462 | 410014.15 | Stomatognathic System - 62%, Exocrine Glands - 14% |
| 473 | 38495.8 | Unclassified/Mixed - 11% |
| 443 | 230889.9 | Unclassified/Mixed - 12%, Germ Cells - 11%, Male Genitalia - 10% |
| 455 | 232928.5 | Unclassified/Mixed - 13% |
| 476 | 242821.5 | Unclassified/Mixed - 13% |
| 417 | 223416.6 | Unclassified/Mixed - 14%, Nervous System - 14%, Endocrine |
| | | System - 12% |
| 429 | 216242.2 | Unclassified/Mixed - 15% |
| 493 | 992317.12 | Unclassified/Mixed - 19%, Exocrine Glands - 14% |
| 412 | 978146.2 | Unclassified/Mixed - 19%, Liver - 15%, Germ Cells - 15% |
| 408 | 196677.1 | Unclassified/Mixed - 24%, Endocrine System - 10% |
| 484 | 985408.1 | Urinary Tract - 12%, Female Genitalia - 12%, Pancreas - 11% |
| 440 | 400104.6 | Urinary Tract - 14%, Pancreas - 13%, Female Genitalia - 11% |
| 472 | 903554.2 | Urinary Tract - 19%, Nervous System - 13% |
| 453 | 1135179.1 | Urinary Tract - 33%, Liver - 21%, Connective Tissue - 17% |
| 507 | 235106.1 | Urinary Tract - 78%, Digestive System - 22% |
| 406 | 366288.5 | widely distributed |
| 409 | 232589.78 | widely distributed |
| 418 | 380601.63 | widely distributed |
| 436 | 480324.16 | widely distributed |
| 437 | 41856.14 | widely distributed |
| 439 | 233720.7 | widely distributed |
| 445 | 1248547.1 | widely distributed |
| 454 | 20434.12 | widely distributed |
| 463 | 1326983.14 | widely distributed |
| 465 | 244999.5 | widely distributed |
| 466 | 239501.1 | widely distributed |
| 470 | 335705.2 | widely distributed |
| 485 | 413491.14 | widely distributed |
| 494 | 200068.22 | widely distributed |
| 513 | 411426.42 | widely distributed |

What is claimed is:

1. A combination comprising a plurality of cDNAs that are differentially expressed in a liver treated with a toxin and are selected from SEQ ID NOs: 1-514 and the complements thereof.

2. The combination of claim 1 wherein each of the cDNAs is differentially expressed at least three-fold.

3. The combination of claim 1 wherein the toxin is selected from acetominophen (APAP), benzo(a)pyrene (BP), clofibrate (CLO), α-naphthylisothiocyanate (ANIT), 4-acetylaminofluorene (4-AAF), hydrazine (Hydra), fenofibrate (Feno), and carbon tetrachloride (CCLA).

4. A combination comprising a plurality of cDNAs that are differentially expressed in a liver disorder and are selected from:
a) SEQ ID NOs: 1-202;
b) SEQ ID NOs: 203-399;
c) SEQ ID NOs: 400-514; and
d) the complement of (a), (b), or (c).

5. The combination of claim 4 wherein the liver disorder is selected from biliary cirrhosis, X-linked adrenoleukodystrophy, Zellweger syndrome, hepatorenal syndrome, hepatitis, and hepatocarcinoma.

6. The combination of claim 4 wherein the liver disorder is biliary cirrhosis.

7. The combination of claim 4 wherein the cDNAs are immobilized on a substrate.

8. A high throughput method for detecting differential expression of one or more cDNAs in a sample containing nucleic acids, the method comprising:
a) hybridizing the substrate of claim 7 with nucleic acids of the sample, thereby forming one or more hybridization complexes;
b) detecting the hybridization complexes; and
c) comparing the hybridization complexes with standards, wherein the differences between the standard and sample hybridization complexes indicates differential expression of cDNAs in the sample.

9. The method of claim 8, wherein the nucleic acids of the sample are amplified prior to hybridization.

10. The method of claim 8, wherein the sample is from a subject with biliary cirrhosis and comparison with a standard defines an early, mid, or late stage of that disease.

11. A high throughput method of screening a plurality of molecules or compounds to identify a ligand which specifically binds a cDNA, the method comprising:
a) combining the combination of claim 1 with the plurality of molecules or compounds under conditions to allow specific binding; and
b) detecting specific binding between each cDNA and at least one molecule or compound, thereby identifying a ligand that specifically binds to each cDNA.

12. The method of claim 11 wherein the plurality of molecules or compounds are selected from DNA molecules, RNA molecules, peptide nucleic acid molecules, mimetics, peptides, transcription factors, repressors, and regulatory proteins.

13. An isolated cDNA selected from SEQ ID NOs: 411, 441, 432, 450, 457, 465, 474, 477, 499, 501, 510.

14. A vector containing the cDNA of claim 13.

15. A host cell containing the vector of claim 14.

16. A method for producing a protein, the method comprising the steps of:
a) culturing the host cell of claim 15 under conditions for expression of protein; and
b) recovering the protein from the host cell culture.

17. A protein or a portion thereof produced by the method of claim 16.

18. A high-throughput method for using a protein to screen a plurality of molecules or compounds to identify at least one ligand which specifically binds the protein, the method comprising:
a) combining the protein of claim 17 with the plurality of molecules or compounds under conditions to allow specific binding; and
b) detecting specific binding between the protein and a molecule or compound, thereby identifying a ligand which specifically binds the protein.

19. The method of claim 18 wherein the plurality of molecules or compounds is selected from DNA molecules, RNA molecules, peptide nucleic acid molecules, mimetics, peptides, proteins, agonists, antagonists, antibodies or their fragments, immunoglobulins, inhibitors, drug compounds, and pharmaceutical agents.

20. A method of using a protein to produce an antibody, the method comprising:
a) immunizing an animal with the protein of claim 17 under conditions to elicit an antibody response;
b) isolating animal antibodies; and
c) screening the isolated antibodies with the protein, thereby identifying an antibody which specifically binds the protein.
